SEQUENCE LISTING (55 sequences)

SEQ ID NO: 1 - DNA, 942 bp, Clostridium acetobutylicum
  source 1..942: mol_type = genomic DNA
atgagaacatatggatttggaatagacattgggggaacaacaataaaaatgggtctattcagagttgatggcactttacttgataagtgggagattcctactagaaaagaggaaagtggaaagtatatattaagtgatatatcaaaagaaataaaagataagatgaaggaaaaaggaatacatagaaatgatgtaattggtgtaggagttggagtaccaggaccagttagcagcgatggaaccgttttaaaatgtgtgaacttaggatggggcatatttaatgtagaagaaactttaagtgaaatgatagatattccagtaaaggctggtaatgatgctaatatagcagctttgggcgaaatgtggcagggaggaggaaaaggctataaaaatgttgttatggttacacttggaacaggcgttggcggaggaattataattaacggcaatataatttctggagcaaatggtgcaggtggagaaataggccatattaaagtagaggaaaatgagaaagatgtttgtggatgtggaaagacaggctgtcttgaacagtatgcctcagcaaatggaattgtaagagaagcaaaaaaacttttaaataatagtagtgaaccttcctctttgagagaaattaaaacggttacttcaaaggatatatttgatgctgcaaaagaaggagataaattagctaatgaacttgttgaagctttaggaaaaaaattaggtaatgcattagccacagtttcatgtgttagtgacccagaggtttttgttgtaggtggaggagtatcaaaggctggaatgattcttataaatgtaattcagaaaaatttcattgaaaaggcatttcatgcatgtgaaggaactaagtttgagcttgcaaggcttggaaatgatgcaggaatttacggcggagtaaaattagtattagagatataa

SEQ ID NO: 2 - DNA, 1158 bp, Clostridium acetobutylicum
  source 1..1158: mol_type = genomic DNA
atgtttgatatagaccagaattccaataagggtaaaattttaaacttgttatatagaaaaagagagcttactaaacttgatatatcaaatgagattggtgtaagtgttccaacagtaatatccaatgtgaatgaacttataagagaaggctttgtagaagaatcaggagtagcaagctctacagggggaaggaaacctataatagtgagctttattccaaatagtagatatgcttttggtgtagatataaatccatatatggcaagagtagttttaactaatcttgacttagaaattaagtatgatgttgagtttttaatagaagagcttaaggatattaaatctataatgaaaaggatatcgcttatagttaaagaggctctggagtttacaaagattgatgagaagaaggtacttggaattggattctcacttcaaggaacagttgatgaggaaaatctagtactcaaaagagctatcaatataggagttaaggactgggattttaaattgtttgaaaacctatttcaatttcctctatttattgaaaacgaagcaaacgctgcagcacttgcagagctaaaaataggtatagcaaaagaggagagaaatcttgtctatatatcagtttcaagcggattaggtactggaatagtgattgagggacagctttataagggaaagaacaagagagctggagaaatgggtcatatgaccttagttcctcatggaaaactgtgcagatgtgggagaaaagggtgctgggaaatgtatgcatctcaaaaagctcttttagatgaatttaatgaattaagtgatgtaaaggttactaagttagagcagttttttaatctaataatgataaaaaataaagttgcagagaaatgtctaaataattacttaaacaatcttgcatcaggtatacaaaatatagttctaatttttgaccctcattatatagttctagggggagaaataagtggatattctgatgtttatttagatagtcttaaggataaaattttcgatgaaaatgagttttataatagaggtgattttaagctattcccatctaggcttaaaaggaattcctcaattttaggagcggctttattgcctatacaaaaagcatttttaattgaacaagatgtaatttga

SEQ ID NO: 3 - DNA, 1089 bp, Clostridium acetobutylicum
  source 1..1089: mol_type = genomic DNA
atgaagcacaaatatgaagaagtagaagaaaaaattatagattgggctgttaaaggaaaatataaaccccatgaaaaaatccccactgaatctgagcttatggaaatgtttagtgtgagcagacataccataagaaaagctataggtgatcttgtagccatgcaatatgtatatagaattcaaggtagtggaatatatatctctgattggacagaaaatagagaatatctaaagaatactaaaaatgtagctgtactaacaacccatatatctaattatattttccctgacataattaagggcatggaggacacgctttattctgaatcctactcacttttactatcttcaactaataacaacgttatgtttgaaaatagtaatttaaaaaacttattagcccacaaaatagatggtctcattttagaacccactaaaagtgcgtaccaaagcccaaatattggatatttaaacaatataatatccaaaaatattcctgttgtaaccctaaatgcctcatatccggagatcaacactcctagcttaaaagtagatgattttaaaggcggaaaactggcaacaaaccatcttatatctttaggtcacagaaaactgatgggtatatttaaggtcgatgattcacaaggcataaacagaatgaatggatttatttcagaatgtcaggaaaataatattccaccatcacctggacaaattttaacttatctctcagaagaaatatcttcatcccttcctgagaaaatagagtatgcactaaaatcagaaaaaaagcctagtggagttttttgctacaatgatgaaatagcctatatagttctaacaattgctaataaattaaatattaaagttcctgaagaactatctatagttggctttgacgattccaatcttgcagtaattatgcagcctaaattaaccaccataactcatcctaaagaacaaatgggtaaagatgctgccaaattaattataaatttaataaataacagtaatcattttgaaactagtgattccattgtttatgaacctaaactaattattagagattcaaccttaaaattgaaataa

SEQ ID NO: 4 - DNA, 1473 bp, Clostridium acetobutylicum
  source 1..1473: mol_type = genomic DNA
atgaataatacaccaaaattaaaattaggaattgttgcagtaagtagagactgctttccaatggaattatcagagaatagaagaaaggcagtagtagatgcttacaatggagaaatcttcgagtgcttaacaactgttgaaaatgaaaaagatatgagaaaagctttaaaagaagtgaaaagtgcaggtgtaaatgcactagtagtatatttaggtaactttggaccagaaactccagaaactttaattgctaaagaatttgatggaccagttatgtttgcagccgcagcagaagagagaggagacaatctagttaatggacgtggagatgcttattgtggaatgctaaatgcaagctataatttagcacttagaaacataaatgcttatattcctgaatatccagtgggaacagcttcagatgttgcaaatatgattaatgaatttgtaccggttgctactgcattaattggattaaagaatttaaagattatttcttttgggccaagacctcaggatttcctagcttgcaatgctcctattaagcagttatataatttaggtgttgaaatagaggaaaattcggagcttgatttgtttgcctcatttaatgaacatgaaaacgattctagaattccagatgtagtaaaagacatggaggaagagttaggtgagggcaataaaatgcctggtattttgccaaagcttgctcaatatgaacttacattactagattgggcagaggagcataaaggatcaagagaatatgttgtgtttgcaaataaatgctggccagcgtttcaaacacaatttggatgtgtgccttgctatgtaaacagtagactaacggctagaggaattccagtatcttgtgaggttgacatttatggagctttaagtgaatacattggaacatgtgtaagtcaagatgttgtaaccttacttgatattaataacacagtaccaaaagatatgtatgaatcagaaattaaaagcaaatttaactatacgttaaaggataccttcatgggttttcactgtggaaacacagcagcatgtaaattaacaagcggaactatgaaaaatcaaatgattatggcaagagctcttgagccaaatcaagagcctaatataactagaggaacgttagagggagatattgtaccaggagaaatcactttcttccgtctacaaagtaatgcagatagtgagttaacagcctatgtagcagaaggagaggttttacctgttaagacacgctcttttggatctattggagtatttgcaattcctcaaatggggagattttaccgccatgtactaattgaaaatagattcccacaccatggtgcagttgcatttggacattttggtaaagcaatttataatttgtttagatatttgggagttaaagaggtaggttttaataggccaaaggaaatgctatataaaacagaaaatccttttgagtaa

SEQ ID NO: 5 - DNA, 1503 bp, Clostridium acetobutylicum
  source 1..1503: mol_type = genomic DNA
gtgaggtatttattaggtatagacgttggaacatcaggaaccaagacagctttgtttgatgaatgtggaaatacaataaaaacttcaacgcatgagtatgagttatttcaaccacaggttggatgggcagaacaaaatcctgaaaattggtggacagcttgtgttaaaggtataagagaagtaattgaaaaaagtaaaattgatcctttagatattaaaggaattggtataagtggacaaatgcatggacttgttttgattgacaaagagtacaaagtaattagaaactccataatttggtgtgatcagagaacagaaaaggaatgtactcagattacagataccataggaaaagaaaaattaattaggataacaggcaatcctgcattaacgggtttcaccctttcaaaactattatgggttaggaataatgaaccagacaattataagagaatatataaggttcttcttcctaaggattatataagatttaaacttactggagtttttgctgccgaagtttcagatgctagtggtactcagatgttagatataaatactagaaattggagtgaagaacttttagatgacttaagaatagataagaacatattacctgatgtatatgaatcagttgttgttagtggatgcgttatagaaaaagcttcaaaagagactaagttagcagttaacactcccgttgtaggtggggcaggagatcaagcagctggagctatagggaatggaatcgttagagaaggtttaatatcaacagtgataggaacttctggagtagtatttgcggctacagatacgcctagatttgatagtaagggaagagttcatactctttgtcatgcagtgcctaataagtggcatataatgggagttactcagggagcaggcttatcattaaattggtttaaaaggacattttgtgcaaaagaaattttagaaagtaaagaggctggaattaatatttatgatttattgaccgaaaaagcatcacaatcaaagcctggttctaatgggataatttatttgccataccttatgggtgaaagaacaccgcacatagatccaaatgtaaaaggagcttttttaggtatatcacttataaataaccacaatgattttgtgcgtagtatattagaaggagtaggctttagtttaaagaattgtctcgatattattgagaatatgaaggttaatattgaggaaatacgagtaagtggtggaggtgcagaaagcagcatatggagacaaatattgtcagatatatttaactatgagcttacaacagtaaaggcatcagaaggaccagcacttggcgtagcaatacttgcaggagttggtgcaggaatatataattccgtggaagaggcttgtgacaaaatagtaaaaggaaacgaaaaggttatgccaaatgcaaatttaatagaagtatattcaaaggtatatgaggtatataattcagcttatcctaaaataaaagatatataa

SEQ ID NO: 6 - DNA, 1410 bp, Clostridium acetobutylicum
  source 1..1410: mol_type = genomic DNA
atgggggaaaacaattcacataagcattctttattattcatagttctaatttcttgtgctgctggacttggtggcttattatatggatacgacactgcagtaatctctggtgctataggctttttaaagaagctttataatctttcacctgctatgcagggctttgttatttcaagtatcatggtcggtggtgtactaggcgttggtttttctggtttcttgggcgatgctattggtaggagaaaagttttaatgcttgctgctgcactatttgctatttccgctgtaatatcttctatatccacttctgcatttatgctcatttttgctcgtatagtaggaggtcttggtataggaatggcttctgccctatctgtaacatatataactgaatgtgcgccgccatctattcgcggtaggctgtcctcactttaccaactatttaccatacttggtatttcaattaccttctttgtaaacttgggaattgtaaatatgggcagcgaaacctggagagtttcaacaggttggagatatatgttagcttgtggaactgttccagccatagtattcttaattacattattctttgttccagaaagtcctcgttttcttgttaaatcaggaaatataaaaaaggctgctgctgttttaaccaaaatcaacggtgctgaaattgcaaagcaggaactagattccatttctaagtctcttgctaccgaaaacgattcttcattaggtcaacttcttcaacctggtcttagaagagctttattaattggtattttcttagctatatttaatcaagctattggaatgaattcaattacttattatggtcctgaaatatttcaaatgataggtttcaaaaataattctagctttcttgcaaccagtgttattggtgttgttgaagttttctcaacaattttagcaatgtttttaatagataaacttggacgtaaaaaattaatggaaataggatctgctgctatggcagtattcatgctattaatcggaacttctttctatataaaattatcaaatggttttgtaatattaatcttcataatatgttttgttgtttccttctgtatttcaatgggacctataccttggattatgattcctgaaatattccctaaccatttgagagctcgtgcaacaggcatagcaactatcttcctttggggtgctaactgggcaattggacaatttactccaatgcttttaaacggcattggtggagcatatactttttggatattctgtggaattaacgtaatatgcttcttagtagttacaactaaggttcctgagacaaaaaacaaaagtctggaagaaatagaaaagttttggattccaaaatcaaaacaaaatgccaaaggaagttcagtaggagcaaaataa

SEQ ID NO: 7 - DNA, 1368 bp, Clostridium acetobutylicum
  source 1..1368: mol_type = genomic DNA
atgaataaaaaaatatctccagcactaatttatttctttggagccttcggtggatttatgtttggatatgatattggaataattaatggtgctttacctggaattaatgcaacttggcacgtaagttcttggttagaaggatttatcacttctggattgtttgttggagctatgataggagcctcattaatggcttcactagcagataggtttggtcgtcgtagaatgattatgtggagtgcaattgtgtttgcacttggtgcattaggttctgccgtttctactagtactaatcttttaatcggtgctcgtgttattttaggagtagctgtaggtggagcttctgctttagttccaatgtatatgggagaaattagccctgctgaaacacgtggaaaactatctggtttaaatcaattaatgataactgttggaatgcttttctcatatggtgtaaattttgcgtttgctggtgcatttgaaggatggcgttggatgcttggaggagctatggtacctgcaatggtactattaattggaacatttatacttccagagtcaccaagatttttagctagaataggaaagacagaattagcaaaacaagtacttcagactttacgttcaaaggaagaggcagaaactgaatatcaagagattattaattcaaaacatactgaaacaggttcttttggagatttatttgcaaaacaggctttgccagctgtaattgcaggctgtgggttaacacttcttcaacaaattcaaggtgcaaacactattttctactattcatcacaaattttatccaatgtttttggatcagcaaatggtggaactattagtactgttggaattggtgtggttctagtattagcaactattgtaactttattggttgtagacaaattcaaacgtcgtacattatttatgactggttctattggaatgggcgcatctctattattagttggattaatttatccatactctgaagctaaacatgcgtgggcaacttggttagtattcttcttcatatgtttatacgttgttttctatgcatactcttgggcagctactacatggattgttgttggagaattattcccaagtaatgttagaggacttgcaacaggtattgcatcagcagtaaactggtttggtaacattttagttgctttattcttcccagtattacttgaaactgtaggtttatctgtaatcttcttcggttttgctgcaatttgtatcataggatttttatttgcaaaatatgttctttatgaaacaaaaggaaaatctttagaagaaattgagacatatttgtacaatcgttctattggaaaagttagaggattaaatgagtag

SEQ ID NO: 8 - DNA, 1377 bp, Clostridium acetobutylicum
  source 1..1377: mol_type = genomic DNA
atgataggaagttttaaaattaaaatgagggaaaaaataggctatgcatctggagatttagcaagtaacttgatatatcaaaccatttcaatttatttgctatttttctatactaacgttttcggcttatctgctggtcaggccggtgtaatgtttcttgtggtaagatttatcgacgcaattaatgacccaattattggaactttagtcgacaaaaccaatacgcgttttggaagatttagaccatacttattatatggagctgctccttttgcagttttagcttttctgtgttttaccactcctaatttttcagcaacaggtaaattaatttacgcttatgttacttacgtaggcctttcaattacttatacttgtataaatgttccttatggtgcattgacatcagctattacagatgataatcaggaaattgttagcttgacctcagtaagaatgttttttgctaatctaggcggtgtaatcgtttcctactttgttcctgtactttcggcatatttcacaaaatcatttggtctttcaggtggttggcaaataactatgagtattctaggtatagcaggtgccttcctattgctattttgtttttcaagtactaaggaaagagtaaaaagcgttaaccaagatcataagattaaattctctgatctctttgaacaattcaaaactaatagacctcttattgtattaagtattttctttgtactgatatttggaataaattcaattaatagttcaataggcatatattacattacctataacgttggtcgtgctgacttagtacaatggtatacggtattagggtctctccctgcatttgtttgtataccactaataccaaaaatcaacagaaaaattggaaaaaagcctttattaattagttctcttttaataacagtgcttggtaccttatctctgttagtaattccaactcatgcagtagcattaatattagtttctcgtgtgattacatcaataggttcattgactgccggagcatttatgtggtcattaattccagaaacaattgaatatggtgaatacacaacaggaaaacgtttaagcggcttaatttatgctattatcggattctttttcaagtgcggaatggcattaggcggagctgtaccaggaataatactcggtaactttgggtatgtagccaataaaacccaaactcctcatgccctaacgggtatacttctaactgccacagtcgttcctgccgtactcatgatattagctttaatagatattaccttctacaacttagatgataaaaaatataatcatattatagctactttaaaagaaagagctaaattaaatagaggggagaatttaaatttatga

SEQ ID NO: 9 - DNA, 1602 bp, Clostridium acetobutylicum
  source 1..1602: mol_type = genomic DNA
atgattaaaaatccaattttaagaggcttcaatcctgatccaagcatctgtcgtgctgacaccgattattacattgctacttcaacctttgaatggtttcctggagttcaaatacatcattctaaggatcttgttaactggcatcttgtagcacatcctttaaacagaacttcacttttagatatgaagggtaatcctaattcaggtgggatatgggctcctgatttaagttatcatgacggaaaattttggctcatatacacagatgtaaaagtaactgatggaatgtggaaggactgtcacaactatttaactacctgtgaaagtgttgatggtgtatggagcgatccaataacattgaatggttctggttttgacgcctctttattccatgataatgatggaaagaagtaccttgtaaatatgtattgggatcagagaacttacaatcataatttttatggaattgttcttcaagagtattctgataaagaaaagaaactaatcggtaaagctaaaattatatataagggcactgatattaagtacacagaaggccctcatatttatcatattggagattactattatctatttactgctgaaggtggcacaacctatgaacattcagaaacagttgcacgttctaaaaatatagatggtccttacgagatagaccctgagtatcctcttttaacttcatggcatgatccacgaaactcattgcaaaaatgcggtcatgcatctcttgtccatactcatactgatgaatggtacttagcccatttagtaggaagacctctacctgttggaaatcaaccagtacttgaacaaagaggctactgtccacttggtcgcgagacttctatacaaagaattgaatgggtagataattggccaagagtcgttggtggtaaacaggggtctgttaatgttgaagcacctaaaattccagaagtaaagtgggagaaaacctacgatgaaaaagataatttcgacagcgacaaattaaatattaactttcaatccttacgtattcctcttacagaaaatatagcatctttaaaagccaaaaaaggaaatttgaggttatatggtaaagaatctttaacgtcaacctttactcaagcatttatagcaagaagatggcaatcatttaaatttgatgcatcaacaagtgtgtcatttagtcctgatacttttcagcaagcagctggattaacctgctattataatacggaaaattggtctactatacaggttacctggaatgaagataaagggcgtgtaattgatattgtttgttgtgataattttcactttgatatgcctttaaaaagcaatgttattccaattcctaaagatgtggagtatattcacttgaaagttgaagtaagagttgaaacatatcagtactcttattctttcgatgggataaactggtcaaaagttcctgctatttttgaatcgagaaagttatccgatgactatgtacaaggtggcggctttttcacaggagcttttgtaggaattaactgcattgatataactggaaataataaacctgctgactttgattatttttgctataaagaattatag

SEQ ID NO: 10 - DNA, 797 bp, synthetic construct
  misc_feature 1..797: note = promoteur du g ne " xylA "
  source 1..797: mol_type = other DNA
tttaattcctccattttattttattgaaatatttttacctattaatcctataaatgctccttctttatctaggttagtatccttatgggcaattaaccatttgtttttagcaaaaagtagagcatcttcaccagtttttttatactctttttctaaaggcaaatttgtatctcttggcaaaaccacaacacatctgaatcccccttcatttacattgcaaggtgcatagtgaagagtagttgcataaacctcaatagcagtaccagctggaactttaaatgctttaattttagaagtactatatgagtaatcttcttcaatatcatctctagagcctaaaagtagtatgaaatctgttactgctatatttatttcagatgaacgatgatattcgactgcatttaataaataattgttaccattgcagtatcctacttgtattggaagctctccatattctctttcctttaattcctttgcaatatcaagctcttcaagttctttaattgatggttcatatactacatcacctggaagtggtgtagatttcattttttctattatctctgtacaatcataatctcttaatatcttgccatattttttgaactccttgtctgtaacacttttaattaccatattctcacactccccatataatttaaattggttttatttaaaaaatatcttaccaatatgatttaaacttctctaataaatatatgtttttaaagtttgtttaataagttttttagataatctactctttaaataaaagcgctcctacttttaactat

SEQ ID NO: 11 - DNA, 225 bp, synthetic construct
  misc_feature 1..225: note = promoteur du g ne " xylB "
  source 1..225: mol_type = other DNA
aatcaaacccccttaattttaaataactttttaaatatcctttaataagtctattttactttttttacctcctacagtcaatatgtttttgtaaactttttcattttatttatataatggcttatatggcactgcatttcacataaaaattgtaataaaaaaaactgcccttaaattttcgagcagtcttttttattcacattctacacataaaatatataatta

SEQ ID NO: 12 - DNA, 25 bp, synthetic construct
  misc_feature 1..25: note = s quence op ratrice " XBS " (XylR-binding site)
  source 1..25: mol_type = other DNA
actttttaaagtgctttttaaaagt

SEQ ID NO: 13 - DNA, 25 bp, synthetic construct
  misc_feature 1..25: note = s quence op ratrice " XBS " (XylR-binding site)
  source 1..25: mol_type = other DNA
actttataaaacaactttataaagg

SEQ ID NO: 14 - DNA, 1467 bp, Clostridium acetobutylicum
  source 1..1467: mol_type = genomic DNA
atgctaaagaataaaaaattagaattttggtttgtagtaggtagtcagaatttatacggtgaagaagcattaaatgcagtaaaaaaagattctaaagaaattgtggattctttaaatgaaagtggaaaattaccatatcctattgtatttaaaactctagctacatcagcagatgaaattaagaatatagttaaagaaataaattatagagatgaagtagcaggtgttattacttggatgcatacattttctcctgctaaaatgtggatagcaggaacaaagcttttacaaaaaccactattgcatctagcaacacaatttaacgaaaatattccttggaaaactattgatatggattatatgaatttacatcaaagtgcacatggtgatagggaatatggatttattaatgctagattaaacaaaaataacaaagttgttgtaggatattggaaggataatcaagttcaaaaggaaattgcagagtggatgcaggttgcttatgggtacgttgcaagtgaaaatataaaggttgcaagatttggggataacatgcgtaatgttgccgtaacagaaggagataaagtagaagcccaaattcaatttggatggacagttgattattttgctattggtgatttagtggctgaaatgaacaaagtttcacaaaaagatatagatgctacttatgaagagtttaaggatatttatatattagatattggagataatgaccctgaattttatgagaatcatgtaaaagaacaaattaagatagagataggtttacgtaattttctagaggcaggtaattatacagcatttacaacaaactttgaggatctttatggaatgaagcaattacctggacttgcagttcaacgtttaaatgctgaaggttatggttttgcaggtgaaggcgattggaaaacagcagcacttaatcgtttatttaaaattatgactgacaacaagaaaactggatttatggaagactatacttatgagctaagtgctggaaatgaaagaattttgggagcacatatgctagaagttgatccaacacttgcagctagtaaaccaagagttgtagttaaaccacttggaattggagataaggaagcaccagcacgtttaatatttgatggagttgtaggtgatggagtagttgtatctatgcttgatttagggacacactatcgtttacttattaacgaagtaaaggcagttaaacctactgaggatgctcctaatttacctgtagcaaagctagtatggcagccacaaccaaactttaaagatgcagttaaagcatggatttatgctggaggtggacatcatactgttgcaaccttagaattaacagttgagcaagtttatgactggagtcgtatggttggtttagaaacaatagttattgatcataataccaatttaagagatattataaaagagacttcaagataa

SEQ ID NO: 15 - DNA, 1467 bp, Clostridium acetobutylicum
  source 1..1467: mol_type = genomic DNA
atgttagaaaataaaaagatggaattttggtttgtagtaggaagtcaacatttatatggtgaagaggctttaaaagaagtaagaaaaaattctgagacaattgtagatgaattaaataaaagtgctaatcttccatataaaataatatttaaagatttagcaacttctgctgataaaataaaggaaataatgaaggaagttaactatagagatgaagtagcaggagttataacttggatgcatacgttttctccagctaaaatgtggatagcaggtacaaagatattacaaaaacctttacttcattttgcaactcaatataatgaaaatattccatggaaaacaatagatatggattatatgaacttacatcaaagtgctcatggagatagagagtatggatttattaatgcaagacttaaaaagcataataaagttgttgtaggatattggaaggataaagaagttcaaaaacaagtttcagattggatgaaggttgctgcaggatatattgcaagtgaaagcataaaagttgcacgttttggtgataacatgcgtaatgttgcagttacagagggagataaagtagaagctcaaatacaattcggatggacagtagattactttggtataggtgatttagttgctgaaatggacaaggtaagccaagatgaaataaataaaacttatgaagaatttaaagatttatatattttagatccaggtgaaaatgatcctgctttctatgagaaacaagttaaagaacaaatcaaaattgaaataggattaagaaggttcttagaaaaaggaaattataatgcatttacaacaaactttgaagatctttatggaatgaaacagttacctggacttgcagtacaacgtttaaatgctgaaggctatggctttgcaggcgaaggagactggaaaactgcagctttagatagattattaaaggttatgactaataatactgctacaggttttatggaagattacacatatgaacttagtcgtggaaatgagaaggcattaggagctcatatgcttgaagttgacccaacttttgcttcagataaaccaaaggttattgttaaaccactaggaattggagataaagaagatccagcacgtttaatctttaatggttcaacaggaaaaggtgtagcagtttcaatgcttgatttaggaacacattatcgtttaataataaacggacttacagcagtgaaaccagatgaagacatgccaaacctaccagttgctaaaatggtatggaaaccagaaccaaacttcattgaaggagttaaatcttggatttatgcaggtggcggacatcatacagtggtttcactagaattaacagtagaacaggtttatgattggagtcgtatggtaggcttggaagctgtaataatagataaggatactaaattaagagatataatagaaaagacaacaaaataa

SEQ ID NO: 16 - DNA, 1605 bp, Clostridium acetobutylicum
  source 1..1605: mol_type = genomic DNA
ttgattatagatgaaaaaagagtacaagaaatcaaaaacggtgaaacatcacttggaatagaatttggttctactcgaattaaagctgtccttattgcaaacgatttctccatattagctagcggaagctttgaatgggagacaagtttagaaaatggggtttggacatattcaatagatgaaatttggaaagggttacaatacagttaccaaaagcttttcgcagaggtaaaggaaaaatatggagtagtgctttctaagattggttcaattggattttcagcaatgatgcacggatatatggcttttgataataagggagagcttctagttccattcagaacttggcgtaatacaatgacagaagaagctgctaaaaagttaacagatttatttaagtttaatattcctgaaagatggagtatagctcatttatatcaagctattcttcgtaatgaaccccatgttgagaaaattgattttttaactactttagcaggctacattcattggcagctaacaggtgaaaaggttttaggtattggtgatgcttcaggaatgttcccaattgatacacaaacacataactatgataaaaacatgttagaaacctttaagaatttaccagaggttaaaaaacattcgttagatttagaaaaacttctaccaaaggtgttactagcaggagagaatgctggagtattaaccttagagggagcaaaaaaacttgatgtaagtggaaatcttcagccgggtattccattgtgtccaccagagggagatgctggtaccggtatggttgctacaaattcagtggcaccgcgcacaggaaatgtttcagcaggcacatcaatttttgctatggttgttcttgaaaaacctcttaatagggttcatcctgaaatagatatagtaacaacaccttcaggcgatttagttgcaatggttcatgcaaataatggttgctcagatttagaggcttgggttaatatttttcagcaatttactgaggctataaatgtagatatgagaaaagatcttctttatatggcactatatactcaagcttttcaaggagatcctgattgtggtggagttcttgcttacaattattttgcaggtgaaaacatcacaggtgtttcagaaggtagacctatggttgtaaggaaggcaaaaagtaattttaatttgccaaactttatgagagcacatttatttacttctttaggtgcattaaaaattggtatggatatattattaaaggatgaagctgttaaacttgataaattacttggtcatggtggactgtttaagacacctattgtcggccaagcagctgtggctggtgcagtaaatgctcctgtgtcagttatggaaacagcaggagaaggtggagcatggggaatcgctttattagctagctatttaagaagtaaggaaaatgattctacattaccagaattcttatcaaaaaatgtttttgcaggttatgtagcagaagaagtacaacctgatccaaaagacgttaaaggttttgaggagtttattgaaagatataaaaagggaattgctatagaacaagcagctgtagagcatttaatctaa

SEQ ID NO: 17 - DNA, 702 bp, Clostridium acetobutylicum
  source 1..702: mol_type = genomic DNA
atgctagaagctttaaaacaagaagtattagaagcaaatcttatgttgcctaagtatgggttagttgtattcacctgggggaatgttagcggtattgatagagaaaaaggtcttatagtcataaaaccaagtggagttgagtatgatcaaatgaaagctagtgatatggtagttgtggatctaaatgggaaggttgtagaaggtgacttaaatccttctagtgatactccaactcatatagtgctatataaagagttttcagatattaagggaattgttcatacacattcaccttgggctacatcttttgcacaagcaggagtttctattccagctgcaggtacaacccatggagattatttttatggtaatattcctgtaacttcaagaatgaccaaagaggaaatagctacagattatgaaaaacaaacaggagatgttatagtaagaacctttagaaaagagaatattaacccaaatgatgttccagctgttcttgtgaatgatcatgctccttttacctggggaactgaccctaagaatgcagtgcataattctgttgtattagaagaggttgctaaaatgacatatcattctttacagcttaatccacataacattgaaatgagtcaggatttgttagataagcattttaaaagaaaacatggtgcaaatgcatattatggtcaaaaaacaaagtaa

SEQ ID NO: 18 - DNA, 984 bp, Clostridium acetobutylicum
  source 1..984: mol_type = genomic DNA
atggaaaatgaaaaaatattaaatcctattattattcaaagagcagatccaatgatttataaacacaatgatggttattattattttacagcatcggtacctgaatatgatcgaatagaagtaaggaaagctaaaacaatagaagggcttagaaatgcagaacctgttgatgtatggagaagacacgaaagtggagaaatgagtaatttaatttgggcaccagaaatacatttcataaatggagcatggtatatatattttgcggcagctcctgataagaatatagaggatgatacctttaaccacagaatgtttgtaatacaaaatgaaaatgaaaatccttttacaggaaattgggtagaaaagggacgaataaaaacggcatgggaatctttctctcttgatgcaactatttttgaacacaatgaaaagctttactatgtgtgggcacaacaggatattaacattaaaggacattcgaatatatatatagcggaaatggaaaatccatggacattgaaaacaaaacccgttatgcttacaaaaccagaattggaatgggagataaaggggttctgggtaaatgaaggtcctgcagtgttaaaaaagaatggaaagatttttataacttattcagctagtgctacagatgtgaattactgcatcggtatgcttacggcagaggaaaatagcaatcttcttgataaaaattcttggacaaaatcacaaactccagtatttaaaacaagtatggaaaaccatcaatatggaccagggcataatagttttacagtttcagaggatggaaaacatgatgtgatagtttatcatgcaaggaattatacggaaatcaaaggggatccgctttatgaccctaataggcatacaagagctcaaattataaactggagagaagatggaacacctgattttggtgtaccagaagttgatagcttagaaattgaaactccagtaaaagcataa

SEQ ID NO: 19 - DNA, 1236 bp, Clostridium acetobutylicum
  source 1..1236: mol_type = genomic DNA
atgaaaatttttagaaaagacttttttaattttggaggattgtttttcttctactttttaatatgggcagtggtcctaacttttttaccaatgtggttaactgatgttgcacatttagatgcaagtaaagctggttttgtgttttcttctatatcactggttgctcttatatatcagccattctttggtgttattcaagataagcttgtgttcaaaaagtatttgtttgcttttgttgcaattttgatgctttttatggggccattttttagctatgcatatatacctcttctccatctaaatatatatgttggagccatcattggaagcgtatatttaagtgcatgtttttatgcaggagtaggtgttgtagaagcgtatatcgaaaaggttagcagaaataacaagtttgagtatggacatgtaaggttatttggttcaattgcaggtgcaacggcttcatttgttgggggaatactgtatataaaaaatcctgtaagtatattttggtttgcgtcgctgtcagcagtagctctttgtattttactctatgtagctaaggttaacgatactggtgaaagcaaagagataaaaaatcaaaaaagtcaaatcaataaggaaattgtgttttctattcttaagatgaaaaaattttggtttttggcattaataataataggaacagcgtgtatatacgatgtttttgatcaacaattccctaattattatgctagctttttttcaagtaaagcagttggaaccagtatatttagtaggcttgtttcatttcaaacgggtttagaggcagttcttatgatttttgctccagcacttgtaaatagaataggtgctaaaaatggattacttttgtttggaaccttaacctttattcgtatttttggaagtgctacctttacaaataccattgtgctgtcaggaattagattaatagcagcttttgagatgccattacttcttgtttcaataatgaaatatataacacatgtatttgatgtgcgcctttcagctaccgtatatcttttagggtttaattttgcaaaacaattagcagttgtaattttttcgtcaatagcagggaatctctattcagctataggttttagatatacttattatttcttaagtttggttgttttagtagttacacttgtaagcgttatcactttgactaatgataagaaaataaaaggattatcttctaatacagctatgtaa

SEQ ID NO: 20 - DNA, 2391 bp, Clostridium acetobutylicum
  source 1..2391: mol_type = genomic DNA
atgcaaagtataataggaaaacataaggatgaaggaaaaatcacaccggagtatctaaagaaaattgatgcatattggcgtgcagctaattttatatctgtaggtcaattgtatttgctagacaatccattgcttagagaacctttaaaaccagaacatctaaaaagaaaagttgttggtcactggggtactattcctggtcaaaactttatttatgctcatcttaaccgtgttattaaaaaatatgatttagatatgatttatgtttctggtccaggtcatggtggacaagtaatggtgtccaattcttatctagatggaacctatagtgaagtttatccaaatgttagtcgtgatttgaatggcttaaaaaagctatgtaaacaattctcttttccaggtggaatttctagccatatggctcctgaaacaccgggttcaataaatgaagggggagaactaggctattctttagcacattcttttggtgctgtttttgataaccctgatttgattactgcttgtgttgttggagacggagaggcagaaacaggacctcttgcaacatcttggcaagcaaataaatttttaaatccagttactgatggagcagtgcttcctattttacatttaaatggatacaaaattagtaaccctactgtgttgtctcgtattcctaaggatgaacttgagaaattctttgaaggaaacggatggaagccttattttgtagaaggtgaagatcctgaaacaatgcataaattaatggcagaaacattagatatagtaacagaagaaattcttaatattcagaaaaatgctcgtgaaaataacgattgttcacgaccaaagtggccaatgattgtattgcgtacaccaaagggatggacaggtccaaaatttgtagatggtgttccaaatgaaggatctttccgtgcacaccaagtaccgcttgcagtagatagatatcatacagaaaacttagatcaattagaagagtggcttaagagttataaaccagaagaattatttgacgaaaactatagactaataccggaacttgaagaattaactccaaagggaaataagagaatggcggctaatttgcatgctaatggtggtttattattacgtgaactacgtacacctgattttcgtgattatgctgtagatgttcctactccagggagcacagttaagcaggatatgattgaacttggaaaatatgtgcgtgatgttgttaaattaaacgaagatactcgtaatttccgtatttttggaccggatgaaactatgtctaatagattatgggcagtttttgaaggtacgaaacgtcaatggttatcagaaattaaagagccaaatgatgaattcttatcgaatgatggacgtattgttgattcaatgctaagcgaacatttatgtgaaggttggttagagggttatcttttaacaggacgtcatggtttctttgcaagttatgaagccttccttcgtattgttgattctatgattactcagcatggtaagtggttaaaggtaacatcacagctaccatggagaaaagatattgcttctttaaatttaatagcaacatctaatgtatggcagcaggatcataatggatatactcatcaagatccaggtttattaggacatattgtggataaaaaacctgaaatagttagagcatatttaccagcagatgccaataccttattagccgtatttgataaatgccttcatactaaacacaagattaatttattagtaacatcaaaacatccaagacaacagtggttaacaatggatcaagcagttaagcatgtagagcaaggaataagcatttgggattgggcaagtaatgacaaaggacaagaacctgatgtagttatagcttcctgtggagatactccaacattagaggctttggcagctgttacaatccttcatgaacatttaccagaattaaaagttcgttttgtaaatgtagtggatatgatgaaattattacctgaaaatgagcatcctcatggcttaagcgataaggattataatgccttatttacaacagataagcctgtaatatttgcattccatggatttgcacatttaataaatcaattaacatatcatcgtgaaaatagaaatttacatgtacatggttatatggaagagggaactattacaacaccatttgatatgcgtgttcaaaataaattagatcgttttaatcttgtaaaagatgtagtagagaatttacctcagcttggaaatcgtggagcacatcttgttcagttaatgaatgataaattagtagaacataaccaatacattcgtgaggttggagaagatttgccagaaataactaattggcagtggcatgtataa

SEQ ID NO: 21 - DNA, 269 bp, synthetic construct
  misc_feature 1..269: note = promoteur d'un g ne " ara "
  source 1..269: mol_type = other DNA
gaaaactcctccttaagatttatatatgtggaaatcaaagagaaatctaagttacaaatgtaaacatatcaattatatataacgttttcatatatgacttataatcatttgatttcctaagggtaatatattttatgaaagcgattacctatataaatcataatatttatgtgtataaatgtcaagaaaaatttggattttatacgtatgaattgtaaatgaaaggaataggaaatcccttgtaggcaaggcaagggactaaatataaa

SEQ ID NO: 22 - DNA, 498 bp, synthetic construct
  misc_feature 1..498: note = promoteur d'un g ne " ara "
  source 1..498: mol_type = other DNA
ttttttctcctataaaataattgttttttataggaatattatatcaaaaatatgtttatttcacaacaatattgatacgtataaattttaataactgtaatattaatttataaattagagtatattaatttataaagttagaattgtaggtagcattataaagacaattacttttaaaagaagttatacttttaagaatttatacgtatgaaaataaaatgtgttgacatttataagtacaaattatattatataagcataatcgttttctaaaaataatgcataatagtaagttaagttatggaagatcatattttgtagttgttactgatttaaattgttagaaaaagaaaacttctaggttttatttttatcaattttatattatgtttttggtaaacgttttacttgcaaattagtataaaagcaattcgagattactgaaaaataacgtatgtaatgagtaattaaaaaataaaagttagggaggagtatttt

SEQ ID NO: 23 - DNA, 173 bp, synthetic construct
  misc_feature 1..173: note = promoteur d'un g ne " ara "
  source 1..173: mol_type = other DNA
acattaataaattaactgttatactaagaaattagtataacagttaactataaaaaatactaaaaaaataaaataggtacgtaccatttttaaaaaatagttgacatgtacgtacaaaagtagtatatttaatataaagagttacttaaatatttcgggaaggggttttaaaa

SEQ ID NO: 24 - DNA, 204 bp, synthetic construct
  misc_feature 1..204: note = promoteur d'un g ne " ara "
  source 1..204: mol_type = other DNA
aagtaaaatttagaagattatcagattaaatcttcattactttttaaaattaatagagatttaatctgattgttaaaaatatatactaagtaattgccgaaaggattgatgttattagaaagtagtataaaaatttatcattaatgaaatcgtttaaaaacaattgtgatagtatttaattaacagattgaggaggaatataaa

SEQ ID NO: 25 - DNA, 20 bp, synthetic construct
  misc_feature 1..20: note = s quence op ratrice " ABS " (AraR-binding site)
  source 1..20: mol_type = other DNA
aaatttatacgtacaaatta

SEQ ID NO: 26 - DNA, 20 bp, synthetic construct
  misc_feature 1..20: note = s quence op ratrice " ABS " (AraR-binding site)
  source 1..20: mol_type = other DNA
aaatttatacgtataaatta

SEQ ID NO: 27 - DNA, 20 bp, synthetic construct
  misc_feature 1..20: note = s quence op ratrice " ABS " (AraR-binding site)
  source 1..20: mol_type = other DNA
taatatgtacgtatatatta

SEQ ID NO: 28 - DNA, 5960 bp, synthetic construct
  misc_feature 1..5960: note = plasmide pGRNA-deltaCA_C1340
  source 1..5960: mol_type = other DNA
agctcggtacccggggatccaccagaaaaaccaacgcctagtacaccaccgaccatgatacttgaaataacaaagccctgcatagcaggtgaaagattataaagcttctttaaaaagcctatagcaccagagattactgcagtgtcgtatccatataataagccaccaagtccagcagcacaagaaattagaactatgaataataaagaatgcttatgtgaattgttttcccccatgaaaactcctccttaagatttatatatgtggaaatcaaagagaaatctaagttacaaatgtaaacatatcaattatatataacgttttcatatatgacttataatcatttgatttcctaagggtaatatattttatgaaagcgattacctatataaatcataatatttatgtgtataaatgtcaagaaaaatttggattttatacgtatgaattgtaaatgaaaggaataggaaatcccttgtaggcaaggcaagggactaaatataaattatttcaattttaattcttctacttcttcatatttgtgcttcatttttttctcctataaaataattgttttttataggaatattatatcaaaaatatgtttatttcacaacaatattgatacgtataaattttaataactgtaatattaatttataaattagagtatattaatttataaagttagaattgtaggtagcattataaagacaattacttttaaaagaagttatacttttaagaatttatacgtatgaaaataaaatgtgttgacatttataagtacaaattatattatataagcataatcgttttctaaaaataatgcataatagtaagttaagttatggaagatcatattttgtagttgttactgatttaaattgttagaaaaagaaaacttctaggttttatttttatcaattttatattatgtttttggtaaacgttttacttgcaaattagtataaaagcaattcgagattactgaaaaataacgtatgtaatgagtaattagtcgacgtcacgcgtccatggagatctcgaggcgtgctagcttataatataatattgtggaatattgaaattaattttactaaaaactattccaatcaaggcagcaataataaaaatagttttatttattggtttagagtacgtgtttttatttttaagtttctttttaatagctgttagaaagaaacttaaaaataaaatatatataataataaatataataaaaatatatataaatttagaaaaagataatatttgataaaacatatttaaagatataatcaaatcctgtaaagatgaaaatgaaaataaaattcctaaaaataagtttaataaagccggaaatttcactagaaatttcactagtttttttaaaaacaataaatataataaagatattattataaaacttaaatttaataataacatcataaggaaaatggattggtttacttcaacggctttattatatgaaaaaaatacaattaaatttaaacataatattacaaaaagtattgttaaaattgaaaaaaattaaatctttagtatctttggaaataatcataatttataagctcctttgattttttatataaattatacattaattatttaatatataaaaactaaaattagtaaatcatctaaatattacaaatgaagaaatattatttgtaaataattatagtaatatattatggaatgaaatagtaaaaaattaaaaagaatcatttaatttagtttaaaatattttaataaaaataataaattaaaacataaaaaatagattaagtttcaattggggacttactctattttttattaagagtttaagaatagttaataattattgaaaatatgatataattttcttagttttacagaaggggaggtgaataagtgagaaacgcattaaagctgaataaaaagaactatatagataatattcatagtagaagtaaagggtggataaccaggagcgttatagataaaaaaggatatagccaatggcactataaatacgctgaattaaaagatttagatatgagtgatgaaaatatctatataactctaaataccttttataagccgtgtaggcgattagaaaatataaaagagttaaatacactgtttatagacttggattattataaaactggcaaaactaaagaccaggtattaatggacttagaaaagaattattttaatcaaagtattcctataccaaactatgtaatagatagtggaagaggaatgtatttaatatggataataaatgcagtacctagtaaagcattaccattatggaaagcggttcaagaatatttatataatcaattaaaatactttggagcagatagacaagccttagatgcaacgagaatattaagagttccaggaagtataaactctaaatcaaaaacagtagtcaatatattagatgagtacgaatatatttatgacttaagagaaatacaaaatggatttttacctgaattaaaaccatatgaaaggaaaaagggtagaccaagcaaaataaattatatttatagagaaagaagtttatattatggaagaatacaagacataataaaactttgtgaactaagagaatatgatttaaaaggacacagagagcttatattatttttatatagatattatctttgtagctttacagaagacattgagaaggcattaaatgatgttttagaacttaatagtatgtttagacaacatttaagtgaaagagaagttataagagcaactagaagtgctgaaagatgttatttagataaaaataagcaatataagtataagaatgaaactcttatagagttattagaaattacagaagaagaacaaaaatatatgacaataataatttctaaaaaggaatataagagaagagaaaatattagaggtaaaaaaaattatcaagagcaattaaaagctaaaggaaaagcaacaaaaaaagaggaattaaatgtattaagaaaaaaaataaaagcccttaaagaaaaaggctttaaaaataaagaaattactctaatgttagaagtaccaataaaaacattagaacgtcatattacgtatatgaaaaaaaatgggcttttataaaggctcattttttatattcttttcttcaaagattatataatataaaaaaatttttttcaaactttaaataaaaaatatttttatatttttttatttttttatttttatatttttttatttttttatttttatatttttttatttttatatttttttatttttatatttttttatttttttatttttttatttttttatttttttatttttttatttttttatttttaccctcattttttttacgcttgtattatagggtactttgtacctgttcttttttttggggaggttgtaaagataattttttactttagttagttccgaaggaacttttattatagtgcccctttatttttttgcgtggcattgaatgtaaaaaattatcactatactagggcgtaaagtaatattacatgtgtctcaaagtgggattaaagcgggattttatagggcgtgtttgtggcttagagtgggattattggaaatttttttgatcctaggttaaatactagtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtaggtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccggggatcgatccccggccgagcgcttagtgggaatttgtaccccttatcgatacaaattccccgtaggcgctagggacctctttagaccggtcagctgtcagacacttatcacattaagtatatactagtattaagctagctttggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcactctatcattgatagagtttgaaactctatcattgatagagtataatatctttgttcattttaaggtcgatgattcacagttttagagctagaaatagcaagttaaaataaggctagtccgttatcaacttgaaaaagtggcaccgagtcggtgctttttttgaagcttg

SEQ ID NO: 29 - DNA, 5939 bp, synthetic construct
  misc_feature 1..5939: note = plasmide pGRNA-deltaCA_C2613
  source 1..5939: mol_type = other DNA
atcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaaatgagggtaaaaataaaaaaataaaaaaataaaaaaataaaaaaataaaaaaataaaaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctcgttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaatttttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttctagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagcacgcctcgagactctatcattgatagagtttgaaactctatcattgatagagtataatatctttgttcatgttacacttggaacaggcgtgttttagagctagaaatagcaagttaaaataaggctagtccgttatcaacttgaaaaagtggcaccgagtcggtgctttttttgaagcttctcgagatctccatggacgcgtgacgtcgacaaagagaaaataagaggaatacaaaagtaaaatgatgttacttaataagaaaattataaatttagttgctaaagagttttatgatatgtatactattgttagaagtttaacatactttttagaaaatcgtgggaaaaatatccaagctattcttcaataagatacataatagtgtaaaataaggagaattagttaagatatgaaaaggatttatctattagaaaatatgctaaacttaaacttttgtgcaaaatccatctaatttgttatatatagtgctgattatataattaaaatgtagtgctatgcatattataagtaggtctactttaaaacaaatgactataaaagataagctttaatgctatatcattattatataaggaggatgttaaatgagaacatatggatttggaatagacattgggggaacaacaataaaaatgggtctattcagagttgatggcactttacttgataagtgggagattcctactagatacggcggagtaaaattagtattagagatataataattatatattttatgtgtagaatgtgaataaaaaagactgctcgaaaatttaagggcagttttttttattacaatttttatgtgaaatgcagtgccatataagccattatataaataaaatgaaaaagtttacaaaaacatattgactgtaggaggtaaaaaaagtaaaatagacttattaaaggatatttaaaaagttatttaaaattaagggggtttgattgtgaggtatttattaggtatagacgttggaacatcaggaaccaagacagctttgtttgatgaatgtggaaatacaataaaaacttcaacgcatgagtatgagttatttcaaccacaggttggatgggcagaacaaaatcctgaaaattggtggacagcttgtgttaaaggtataagagaagtaattgaaaaaagtaaaattgatcctttagatattaaaggaattggtataagtggacaaatgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaaagctagcttaatactagtatatacttaatgtgataagtgtctgacagctgaccggtctaaagaggtccctagcgcctacggggaatttgtatcgataaggggtacaaattcccactaagcgctcggccggggatcgatccccgggtacgtacccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaactagtatttaacctagg

SEQ ID NO: 30 - DNA, 5960 bp, synthetic construct
  misc_feature 1..5960: note = plasmide pGRNA-deltaCA_C3673
  source 1..5960: mol_type = other DNA
agctcggtacccggggatccagaaagacttgttacatctctaagttttccttccctttggaatatattataagcggtttgtcctggataaacttgaaatatatccggtgcaacatcatttctaaccctatcatttattatagttgcagcttttgatggcgactcaaccttcactttatatcccgtttctttttcaaaatctgaagctatatcttgaaaagtattaagcatttcaaatttttctacaaaaaaatcaacatcataatcatcattttttatagaaacgcaccctacagtactaaaaattatacaagttataatagaaaataaacataattttttgagccatcccattattttttaccccctatttcactagttaaattaaatcgtttacataaatgattttaagaagactttcctcaataagtctcctatgctttttctatcatctattaaaatacaatactatttatatttacctgtgtttactaatttcaaattacatcttgttcaattaagtctatatcaaacatatcgcaccaaccttaatcactagtattttactttaccttcatattgtaccatatactatatccccacaaaagcctttataaagttgttttataaagtatatatactatgtatattttttcttatatagttttaatacaaaaatgaagtaaagatatcaattatatatctttacttcacttacgttatgcctctttttttatatttttaaacatctcagaatagcaataattcataatatcgcttctttttattatgcctataaatacattattatcatcaacaacaggaacaaaattttgacttacagattttgatataagatcctcaatatcagaatctatatgcacaggtctattattcatgtgtctcggtatatccttaattgaaaccttatttgtcgtttgaaaatcaatctccggagtattttttaatttccacaataaatcgccttctgttaaagttcctatatatttcccattatcatgtcgacgtcacgcgtccatggagatctcgaggcgtgctagcttataatataatattgtggaatattgaaattaattttactaaaaactattccaatcaaggcagcaataataaaaatagttttatttattggtttagagtacgtgtttttatttttaagtttctttttaatagctgttagaaagaaacttaaaaataaaatatatataataataaatataataaaaatatatataaatttagaaaaagataatatttgataaaacatatttaaagatataatcaaatcctgtaaagatgaaaatgaaaataaaattcctaaaaataagtttaataaagccggaaatttcactagaaatttcactagtttttttaaaaacaataaatataataaagatattattataaaacttaaatttaataataacatcataaggaaaatggattggtttacttcaacggctttattatatgaaaaaaatacaattaaatttaaacataatattacaaaaagtattgttaaaattgaaaaaaattaaatctttagtatctttggaaataatcataatttataagctcctttgattttttatataaattatacattaattatttaatatataaaaactaaaattagtaaatcatctaaatattacaaatgaagaaatattatttgtaaataattatagtaatatattatggaatgaaatagtaaaaaattaaaaagaatcatttaatttagtttaaaatattttaataaaaataataaattaaaacataaaaaatagattaagtttcaattggggacttactctattttttattaagagtttaagaatagttaataattattgaaaatatgatataattttcttagttttacagaaggggaggtgaataagtgagaaacgcattaaagctgaataaaaagaactatatagataatattcatagtagaagtaaagggtggataaccaggagcgttatagataaaaaaggatatagccaatggcactataaatacgctgaattaaaagatttagatatgagtgatgaaaatatctatataactctaaataccttttataagccgtgtaggcgattagaaaatataaaagagttaaatacactgtttatagacttggattattataaaactggcaaaactaaagaccaggtattaatggacttagaaaagaattattttaatcaaagtattcctataccaaactatgtaatagatagtggaagaggaatgtatttaatatggataataaatgcagtacctagtaaagcattaccattatggaaagcggttcaagaatatttatataatcaattaaaatactttggagcagatagacaagccttagatgcaacgagaatattaagagttccaggaagtataaactctaaatcaaaaacagtagtcaatatattagatgagtacgaatatatttatgacttaagagaaatacaaaatggatttttacctgaattaaaaccatatgaaaggaaaaagggtagaccaagcaaaataaattatatttatagagaaagaagtttatattatggaagaatacaagacataataaaactttgtgaactaagagaatatgatttaaaaggacacagagagcttatattatttttatatagatattatctttgtagctttacagaagacattgagaaggcattaaatgatgttttagaacttaatagtatgtttagacaacatttaagtgaaagagaagttataagagcaactagaagtgctgaaagatgttatttagataaaaataagcaatataagtataagaatgaaactcttatagagttattagaaattacagaagaagaacaaaaatatatgacaataataatttctaaaaaggaatataagagaagagaaaatattagaggtaaaaaaaattatcaagagcaattaaaagctaaaggaaaagcaacaaaaaaagaggaattaaatgtattaagaaaaaaaataaaagcccttaaagaaaaaggctttaaaaataaagaaattactctaatgttagaagtaccaataaaaacattagaacgtcatattacgtatatgaaaaaaaatgggcttttataaaggctcattttttatattcttttcttcaaagattatataatataaaaaaatttttttcaaactttaaataaaaaatatttttatatttttttatttttttatttttatatttttttatttttttatttttatatttttttatttttatatttttttatttttatatttttttatttttttatttttttatttttttatttttttatttttttatttttttatttttaccctcattttttttacgcttgtattatagggtactttgtacctgttcttttttttggggaggttgtaaagataattttttactttagttagttccgaaggaacttttattatagtgcccctttatttttttgcgtggcattgaatgtaaaaaattatcactatactagggcgtaaagtaatattacatgtgtctcaaagtgggattaaagcgggattttatagggcgtgtttgtggcttagagtgggattattggaaatttttttgatcctaggttaaatactagtttttagttacagacaaacctgaagttaactatttatcaattcctgcaattcgtttacaaaacggcaaatgtgaaatccgtcacatactgcgtgatgaacttgaattgccaaaggaagtataattttgttatcttctttataatatttccccatagtaaaaataggaatcaaataatcatatcctttctgcaaattcagattaaagccatcgaaggttgaccacggtatcatagatacattaaaaatgttttccggagcatttggctttccttccattctatgattgtttccataccgttgcgtatcactttcataatctgctaaaaatgatttaaagtcagacttacactcagtccaaaggctggaaaatgtttcagtatcattgtgaaatattgtatagcttggtatcatctcatcatatatccccaattcaccatcttgattgattgccgtcctaaactctgaatggcggtttacaatcattgcaatataataaagcattgcaggatatagtttcattcccttttcctttatttgtgtgatatccactttaacggtcatgctgtaggtacaaggtacacttgcaaagtagtggtcaaaatactcttttctgttccaactatttttatcaattttttcaaataccatctaagttccctctcaaattcaagtttatcgctctaatgaacaaagatattataccacatttttgtgaatttttcaacttgcccacttcgactgcactcccgacttaataacttcttgaacacttgccgaaaaagaaaaactgccgggtacgtacccggggatcgatccccggccgagcgcttagtgggaatttgtaccccttatcgatacaaattccccgtaggcgctagggacctctttagaccggtcagctgtcagacacttatcacattaagtatatactagtattaagctagctttggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcactctatcattgatagagtttgaaactctatcattgatagagtataatatctttgttcatggagtagcaagctctacagggttttagagctagaaatagcaagttaaaataaggctagtccgttatcaacttgaaaaagtggcaccgagtcggtgctttttttgaagcttg

SEQ ID NO: 31 - DNA, 36 bp, synthetic construct
  misc_feature 1..36: note = amorce CA_C2613_LHA_Fwd
  source 1..36: mol_type = other DNA
aaaaaagaattcatttgtccacttataccaattcct

SEQ ID NO: 32 - DNA, 30 bp, synthetic construct
  misc_feature 1..30: note = amorce CA_C2613_LHA_Rev
  source 1..30: mol_type = other DNA
tcctactagatacggcggagtaaaattagt

SEQ ID NO: 33 - DNA, 30 bp, synthetic construct
  misc_feature 1..30: note = amorce CA_C2613_RHA_Fwd
  source 1..30: mol_type = other DNA
ctccgccgtatctagtaggaatctcccact

SEQ ID NO: 34 - DNA, 39 bp, synthetic construct
  misc_feature 1..39: note = amorce CA_C2613_RHA_Rev
  source 1..39: mol_type = other DNA
aaaaaagtcgacaaagagaaaataagaggaatacaaaag

SEQ ID NO: 35 - DNA, 24 bp, synthetic construct
  misc_feature 1..24: note = amorce gRNA_CA_C2613_Fwd
  source 1..24: mol_type = other DNA
tcatgttacacttggaacaggcgt

SEQ ID NO: 36 - DNA, 24 bp, synthetic construct
  misc_feature 1..24: note = amorce gRNA_CA_C2613_Rev
  source 1..24: mol_type = other DNA
aaacacgcctgttccaagtgtaac

SEQ ID NO: 37 - DNA, 37 bp, synthetic construct
  misc_feature 1..37: note = amorce CA_C3673_LHA_Fwd
  source 1..37: mol_type = other DNA
aaaaaaggatccagaaagacttgttacatctctaagt

SEQ ID NO: 38 - DNA, 41 bp, synthetic construct
  misc_feature 1..41: note = amorce CA_C3673_LHA_Rev
  source 1..41: mol_type = other DNA
tgatatagacttaattgaacaagatgtaatttgaaattagt

SEQ ID NO: 39 - DNA, 37 bp, synthetic construct
  misc_feature 1..37: note = amorce CA_C3673_RHA_Fwd
  source 1..37: mol_type = other DNA
gttcaattaagtctatatcaaacatatcgcaccaacc

SEQ ID NO: 40 - DNA, 40 bp, synthetic construct
  misc_feature 1..40: note = amorce CA_C3673_RHA_Rev
  source 1..40: mol_type = other DNA
aaaaaagtcgacatgataatgggaaatatataggaacttt

SEQ ID NO: 41 - DNA, 24 bp, synthetic construct
  misc_feature 1..24: note = amorce gRNA_CA_C3673_Fwd
  source 1..24: mol_type = other DNA
tcatggagtagcaagctctacagg

SEQ ID NO: 42 - DNA, 24 bp, synthetic construct
  misc_feature 1..24: note = amorce gRNA_CA_C3673_Rev
  source 1..24: mol_type = other DNA
aaaccctgtagagcttgctactcc

SEQ ID NO: 43 - DNA, 20 bp, synthetic construct
  misc_feature 1..20: note = amorce CA_C2613_verif_Fwd
  source 1..20: mol_type = other DNA
tcggcagcaaaaactccagt

SEQ ID NO: 44 - DNA, 20 bp, synthetic construct
  misc_feature 1..20: note = amorce CA_C2613_verif_Rev
  source 1..20: mol_type = other DNA
gcaattgctggcggaatgaa

SEQ ID NO: 45 - DNA, 26 bp, synthetic construct
  misc_feature 1..26: note = amorce CA_C3673_verif Fwd
  source 1..26: mol_type = other DNA
tacttcatacatatctaacgcactct

SEQ ID NO: 46 - DNA, 26 bp, synthetic construct
  misc_feature 1..26: note = amorce CA_C3673_verif Rev
  source 1..26: mol_type = other DNA
aaataacttctactatgagacaagca

SEQ ID NO: 47 - DNA, 6102 bp, synthetic construct
  misc_feature 1..6102: note = plasmide pBANak
  source 1..6102: mol_type = other DNA
aaaatataaaattagaaagctctctccattagaatgttggagactccaagcatttgatgacgaagattttgaaaaagcttttgcagcaggaataagtaactcacaattatacaagcaagccggtaattcaattacagttagtgtgcttgagtctatatttcaagaattaatacatacatatgttaataaagaatctgaataaaatttgtcttttaaacaaatgcaaaataagagggattgagaggtgaggaagatggatagttacccagagtctttaaaaagagagacagaggagattaaagttcagcacactgagacttgttgagttgaattcggtcagtgcgtcctgctgatgtgctcagtatctctatcactgatagggatgtcaatctctatcactgatagggactcgaggtgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgccgaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggcgcggccgcgcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagcatcgatgaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatatttggtgaaaagtgttttcggactcgaagatgtatcgccgactataaaaagtcaaaacgcaaatcttcctcctggttatcccggacaccctaaagacgccgatcaaatctgcaaccgaccgattagccaacaaaagcgccccgccgtttcaccgttcgcgaatgtctccgaatccaatctgcgccagacacttacgtcctgccagacgatatttccttaacggcgcagtatcggattagtcggtaacggaattgcttcgcgtgttgcgtggtatatcgggcgggcgttgcggatcaactttcatctaactgtaagtaattatgatagataagatggttgaaatattacataaaagatcaattttatttagagtaaaaataaaatatatggaggttgtttgttgagtaaactacgtgtaatgagcctttttagtggaattggtgcatttgaagctgcattaagaaacataggcgttgattatgaactgattggctttagtgaaatagataaatatgcaatcaaatcttattgtgcaattcataatgtaagtgaaacattgaatgttggagacataagtaaagctaagaaagataatattccatattttgatttgttaacaagtggatttccttgtcccactttttcagtagccggcggccgtgatggtatggaatataagtgtagcaattgttcgcacgagcatttaattacttatgaggattataagaagggagtcaaatgcccgaagtgtgaagctgtttctaaggctaaagatgaacggggaacgctcttttttgaaacagctttgttagcagaagagaagaagccgaaatttgtgatattagaaaatgtaaaagggctaattaatagtggcaatggacaagtgttaagaataattagtgagactatgaacaatataggctataggattgacctagagctacttaattcaaagtttttcaatgttccacagaatcgtgaacgggtatacataattggcattagagaagatttggttgaaaatgaacaatgggttgtaggtcagaagcgaaacgatgttctgagtaaaggtaaaaagagattgcaagaaataaatataaagagttttaattttaaatggcctttacaagacactgtcacaaagaggttgagagaaattcttgaggattttgttgatgagaagtattacttaaatgaagaaaagacgaagaaacttgttgagcaactaggaaccgcaccgctacaaaaacaagaagtaagggagccgttaatggtgggacacgtggatttaaaaggtcacgatgccatcaaaagagtttactcgcctgaaggactttcaccgacattaactacaatgggcggcggtcacagagaacctaaaatcgcagaaaagcagaaagaagtaagggcagtgttaacgccagaaagagaagagaaaagacagaatggaagacgttttaaagaaaacggagaaccggctttcacggttaatacaattgatcgacatggtgtagcaatcggagaataccc

SEQ ID NO: 48 - DNA, 35 bp, synthetic construct
  misc_feature 1..35: note = amorce CA_C1340_LHA_Fwd
  source 1..35: mol_type = other DNA
aaaaaaggatccaccagaaaaaccaacgcctagta

SEQ ID NO: 49 - DNA, 48 bp, synthetic construct
  misc_feature 1..48: note = amorce CA_C1340_LHA_Rev
  source 1..48: mol_type = other DNA
agaagtagaagaattaaaattgaaataatttatatttagtcccttgcc

SEQ ID NO: 50 - DNA, 38 bp, synthetic construct
  misc_feature 1..38: note = amorce CA_C1340_RHA_Fwd
  source 1..38: mol_type = other DNA
tttcaattttaattcttctacttcttcatatttgtgct

SEQ ID NO: 51 - DNA, 43 bp, synthetic construct
  misc_feature 1..43: note = amorce CA_C1340_RHA_Rev
  source 1..43: mol_type = other DNA
aaaaaagtcgactaattactcattacatacgttatttttcagt

SEQ ID NO: 52 - DNA, 24 bp, synthetic construct
  misc_feature 1..24: note = amorce gRNA_CA_C1340_Fwd
  source 1..24: mol_type = other DNA
tcattttaaggtcgatgattcaca

SEQ ID NO: 53 - DNA, 24 bp, synthetic construct
  misc_feature 1..24: note = amorce gRNA_CA_C1340_Rev
  source 1..24: mol_type = other DNA
aaactgtgaatcatcgaccttaaa

SEQ ID NO: 54 - DNA, 23 bp, synthetic construct
  misc_feature 1..23: note = amorce CA_C1340_verif_Fwd
  source 1..23: mol_type = other DNA
cctaccaatagcatcgcccaaga

SEQ ID NO: 55 - DNA, 25 bp, synthetic construct
  misc_feature 1..25: note = amorce CA_C1340_verif_Rev
  source 1..25: mol_type = other DNA
ttctagcataaaatactcctcccta

(19) United States
(12) Patent Application Publication
(10) Pub. No.: US 2026/0275271 A1
(43) Pub. Date: Sep. 17, 2026

(54) CLOSTRIDIUM BACTERIA MODIFIED TO ASSIMILATE SEVERAL SUGARS SIMULTANEOUSLY, PREPARATION AND USES THEREOF

(71) Applicant: IFP ENERGIES NOUVELLES, RUEIL MALMAISON (FR)

(72) Inventors: ALEXANDRE DELAROUZEE, RUEIL MALMAISON CEDEX (FR); NICOLAS LOPES FERREIRA, RUEIL MALMAISON CEDEX (FR); FRANÇOIS WASELS, RUEIL MALMAISON CEDEX (FR)

(21) Appl. No.: 18/878,677

(22) PCT Filed: Jun. 26, 2023
(86) PCT No.: PCT/FR2023/050965

§ 371 Date: Dec. 24, 2024

(30) Foreign Application Priority Data
Jun. 27, 2022 (FR) FR2206411

(51) Int. Cl. C12N 1/205; C07K 14/33; C12P 7/00; C12R 1/145

(52) U.S. Cl. CPC C12N 1/205; C07K 14/33; C12P 7/00; C12P 2203/00; C12R 2001/145

(57) ABSTRACT

The present invention relates to the genetic modification of bacteria of the genus Clostridium, typically solventogenic bacteria of the genus Clostridium. The invention also relates to methods, tools and kits enabling the elimination or modification of sequence(s) encoding, or controlling the transcription of sequence(s) encoding, the transcriptional repressors XylR and/or AraR, to the genetically modified bacteria obtained and to uses thereof, in particular for simultaneously fermenting at least one hexose and one pentose or the carbon-based elements of a substrate comprising at least two carbon-based elements chosen from glucose, arabinose, xylose, mannose and galactose.

The present invention relates to the genetic modification of bacteria of the genus Clostridium, typically solventogenic bacteria of the genus Clostridium. The invention also relates to methods, tools and kits enabling the elimination or modification of sequence(s) encoding, or controlling the transcription of sequence(s) encoding, the transcriptional repressors XylR and/or AraR, to the genetically modified bacteria obtained and to uses thereof, in particular for simultaneously fermenting at least one hexose and one pentose or the carbon-based elements of a substrate comprising at least two carbon-based elements chosen from glucose, arabinose, xylose, mannose and galactose.

### TECHNOLOGICAL BACKGROUND

The Clostridium genus contains Gram-positive, strict anaerobic, spore-forming bacteria belonging to the Bacillota phylum. Clostridia are an important group for the scientific community for several reasons. The first is that a certain number of serious diseases (e.g. tetanus, botulism) are due to infections by pathogenic members of this family (John & Wood, 1986). The second is the possibility of using “acidogenic” or “solventogenic” strains in biotechnology. These non-pathogenic Clostridia naturally have the ability to transform a wide variety of sugars to produce chemical species of interest, and in particular acetone, butanol and ethanol (John & Wood, 1986) during what is known as ABE fermentation. The bacterium Clostridium acetobutylicum is today considered as a model representative of solventogenic Clostridium.

Solventogenic Clostridium are capable of assimilating a wide variety of carbon sources, which is an asset for ABE fermentation processes (Keis, Shaheen and Jones 2001). In theory, this versatility allows them to proliferate efficiently on a wide range of substrates, notably coproducts from the agro-industry (Qureshi et al. 2008; Ezeji, Qureshi and Blaschek 2007; Ezeji and Blaschek 2008; Qureshi et al. 2006). By virtue of the development of industrial fermentation processes, and more recently of green chemistry, the incorporation of plant biomass into industrial processes is becoming increasingly widespread. However, a categorization of plant substrates has rapidly spread throughout the industry, contrasting two “generations” of industrial substrates, based on environmental and human considerations.

The “first-generation” (1G) substrates collate all carbon sources derived from plant reserve organs. In particular, 1G substrates may be derived from corn or wheat, but also from sugar cane or beet. Consisting predominantly of sucrose and starch, their fermentation on an industrial scale is currently very efficient. On the other hand, the use of these substrates is controversial, since they require the use of arable land and commodities exploited by the agrifood industry.

In contrast to this paradigm, “second-generation” (2G) substrates collate all substrates derived from plant structural organs, or from agro-industrial coproducts. In so doing, this approach helps to reduce tensions between the energy and food sectors. However, their use is far less straightforward than for 1G substrates. This is due to the predominant carbon source of these substrates: lignocellulose, a complex and recalcitrant polymer matrix. Its degradation into fermentable sugars requires more or less costly pretreatments, which may generate microbial growth inhibitors.

Due to the complex nature of the different fractions from which they are derived, hemicellulose hydrolysates contain a variety of carbon sources. These sources predominantly include xylose and glucose, accompanied by a mixture of minor sugars such as galactose, mannose and arabinose. All these sugars are fermentable by solventogenic Clostridium species. For example, Clostridium cellulovorans DSM 743B is described by Wen et al. (2020) as a strain that can be manipulated to optimize the production of n-butanol from lignocellulose, and WO 2013/159710 describes a modified C. beijerinckii bacterium with enhanced capacity for consumption of xylose and/or arabinose. In practice, however, a biological process known as Catabolic Repression (CR) prevents the optimal and simultaneous use of all these carbon sources. In particular, catabolic repression prevents the efficient metabolization of xylose contained in lignocellulose hydrolysates. The bacterium C. acetobutylicum in particular is susceptible to this repression phenomenon (Qureshi et al. 2006; Grimmler et al. 2010). Hu et al. (2011) report that inactivation of the xylR gene (CA_C2613) in C. acetobutylicum strain ATCC 824 results in faster xylose use, but does not affect the total amount of xylose fermented. WO 2013/159710 advises those skilled in the art against inactivating xylR (CAC3673) in C. acetobutylicum.

### SUMMARY OF THE INVENTION

In the context of the present invention, the inventors describe bacteria of the Clostridium genus, typically solventogenic bacteria, which are capable of simultaneously consuming different carbon sources, in particular different sugars. In particular, they describe for the first time a C. acetobutylicum bacterium that is capable of simultaneously consuming glucose and a pentose, preferentially glucose and arabinose.

In particular, the inventors describe a genetically modified bacterium belonging to the genus Clostridium, in particular a solventogenic bacterium, which does not express the transcriptional repressors XylR and/or AraR or which expresses non-functional versions thereof. This genetically modified bacterium is a bacterium not expressing the xylR and/or araR gene products, in particular the CA_C2613, CA_C3673 and/or CA_C1340 gene products, or expressing non-functional versions of said products.

A particular bacterium described herein by the inventors does not express the transcriptional repressor XyIR or expresses a non-functional version thereof. This genetically modified bacterium is a bacterium not expressing xylR gene products, in particular CA_C2613 and/or CA_C3673 gene products, or expressing non-functional versions of said products.

Another particular bacterium described herein by the inventors does not express the AraR transcriptional repressor or expresses a non-functional version thereof. This genetically modified bacterium is one which does not express the araR gene product, in particular the CA_C1340 gene product, or which expresses a non-functional version of said product.

Another preferred genetically modified bacterium according to the invention is a bacterium not expressing the transcriptional repressors XyIR and AraR or expressing non-functional versions thereof. This genetically modified bacterium is a bacterium not expressing the xylR and araR gene products, in particular the CA_C2613, CA_C3673 and CA_C1340 gene products, or expressing non-functional versions of said products.

Particularly preferred genetically modified bacteria according to the invention correspond to the strain identified in the present description as IFP 968 as registered on 13 Jun. 2022 under deposit number LMG P-32703 at the BCCM-LMG collection (also identified herein as ΔCA_C1340 ΔCA_C2613 ΔCA_C3673 or ΔaraR ΔxylR1 ΔxylR2), and to the strain identified in the present description as IFP 967 as registered on 13 Jun. 2022 under deposit number LMG P-32702 at the BCCM-LMG collection (also identified in the present text as ΔCA_C1340 or ΔaraR), which may be used to prepare a strain equivalent to the strain IFP 968. The invention is also directed towards any derived bacterium, clone, mutant or genetically modified version thereof.

The inventors also describe a process for producing a recombinant bacterium as described herein, in particular a process comprising the deletion or inactivation of the xylR and/or araR genes so as to prevent or reduce the expression of functional XylR and/or AraR proteins, and the genetically modified bacteria that may be obtained by this process, of which IFP 967 and IFP 968 are examples.

The inventors also describe the use of a genetically modified bacterium belonging to the genus Clostridium, characterized in that it does not express the xylR or araR gene product, or in that it expresses a non-functional version of the xylR or araR gene, such as the strain IFP 967, for preparing a genetically modified bacterium according to the invention not expressing the xylR and araR gene products, or expressing non-functional versions thereof.

The inventors moreover describe the use of a bacterium according to the invention for producing a solvent, a sugar or a biobased molecule, for example from i) a lignocellulosic biomass, ii) the product of a particular dedicated crop such as an energy crop, perennial forage crop, herbaceous crop or shrub crop, or iii) 1G substrate(s) derived from one or more sugar-yielding, cereal or oil-yielding plants, or iv) a mixture of such carbon sources.

The inventors also describe the use of bacteria according to the invention for simultaneously fermenting (or in other words “co-fermenting”) at least one hexose and one pentose, or the carbon-based elements of a substrate comprising at least two carbon-based elements chosen from glucose, arabinose, xylose, mannose and galactose. Such bacteria may advantageously be used to produce a solvent or a mixture of solvents, in particular on an industrial scale.

The invention also relates to a fermentation process involving the use of a genetically modified bacterium as described herein.

Finally, the inventors describe kits, in particular a kit for transforming and preferably genetically modifying a bacterium belonging to the genus Clostridium, and a kit for producing a solvent, a sugar or a biobased molecule using a bacterium belonging to the genus Clostridium, said kit comprising i) a genetically modified bacterium belonging to the genus Clostridium according to the invention and ii) a medium, in particular a rich culture medium, preferably an RCM-type culture medium, more preferentially a GAPES-type culture medium, even more preferentially a CGM-type culture medium, containing at least glucose and a pentose, preferentially glucose and arabinose and/or xylose.

### DETAILED DESCRIPTION OF THE INVENTION

Although used in industry for over a century, knowledge regarding bacteria belonging to the Clostridium genus, in particular solventogenic bacteria, remains very limited. A major constraint encountered by manufacturers relates to their inability to simultaneously use several different sources of carbon, and thus to efficiently metabolize different types of substrates, in particular different types of sugars.

Despite the difficulties, well known to those skilled in the art, encountered in genetically modifying bacteria belonging to the Clostridium genus, the inventors have succeeded, for the first time in the context of the present invention, in obtaining a C. acetobutylicum bacterium that is capable of simultaneously using, i.e. co-assimilating, co-metabolizing or co-fermenting (these terms being used interchangeably in the present text) several different sources of carbon, in particular from biomass, notably plant biomass, for example lignocellulosic biomass.

A “raw biomass”, also known as “native biomass”, which is preferred in the context of the invention is a plant biomass, in particular a lignocellulosic biomass. The latter essentially comprises three natural constituents (polymers) present in varying amounts depending on its origin: cellulose (about 35-50% by dry mass of the biomass), which is a polysaccharide essentially consisting of hexoses; hemicellulose (about 20-30% by dry mass of the biomass), which is a polysaccharide essentially consisting of pentoses; and lignin (about 15-25% by dry mass of the biomass), which is a structurally complex, high-molecular-weight polymer formed from aromatic alcohols linked via ether bonds. These different molecules are responsible for the intrinsic properties of the plant wall and organize themselves in a complex entangled network. Of these three basic polymers that incorporate lignocellulosic biomass, cellulose and hemicellulose are those that allow the production of 2G sugary liquor and 2G alcohol.

Non-limiting examples of lignocellulosic biomass include, for example, agricultural residues (notably straw waste, corn cobs, sugarcane bagasse, etc.), but also forestry residues, sawmill residues (typically wood chips) or any other type of ligneous residue of forestry, industrial, urban or household origin. Cellulosic biomass may also be the product of a particular dedicated crop such as an energy crop, for example dedicated to the cultivation of annual plants (“whole plant” use, for example of wheat or corn). It may also be, for example, a crop of perennial forage plants (for example fescue, cocksfoot or ryegrass), a crop of herbaceous plants (for example miscanthus or switchgrass), or a shrub crop (short or very short rotation coppice, for example willow, poplar, chestnut or eucalyptus).

Another biomass that may be used in the context of the invention may be a 1G substrate or a mixture of several 1G substrates from sugar-yielding plants (for instance sugar cane or sugar beet), cereal plants (for instance corn or wheat) and/or oil-yielding plants (for instance soybean, rapeseed or palm). In the context of the invention, biomass may be used alone or as a mixture, for example of several different types of biomass. The amount of water contained in the raw biomass is between 1% and 70% of the total mass of raw biomass.

To this end, the inventors have thus advantageously succeeded in genetically modifying a solventogenic bacterium, the reference strain C. acetobutylicum. In particular, they have made it capable of fermenting two carbon-based elements of different nature, for example at least one hexose and one pentose, or the carbon-based elements of a substrate comprising at least two distinct carbon-based elements chosen from glucose, arabinose, xylose, mannose and galactose, preferably chosen from glucose, arabinose and xylose, even more preferentially glucose and arabinose.

In C. acetobutylicum, genes linked to xylose catabolismare organized in the form of a regulon (Gu et al. 2010). The main operon involved in xylose assimilation is the CA_C2610-CA_C2612 locus, which contains the genes encoding xylose isomerase XylA (CA_C2610) and xylulokinase XylB (CA_C2612) (Gu et al. 2010). The genes encoding the permeases predicted to be involved in xylose import, AraE1 (CA_C1339) and XyIT (CA_C1345), are located at a second locus (CA_C1339-CA_C1349) also containing genes linked to arabinose assimilation. A third operon (CA_C3451-CA_C3452) contains the genes encoding the XynT and XynB proteins, corresponding respectively to a hypothetical permease and a 1,4-β-D-xylosidase.

With the exception of xylT, all these genes have XylR-binding-site (XBS) operator sequences in their promoter or coding sequence (Gu et al. 2010). Specifically, the presence of XBS has the effect of placing these genes under the control of a xylose repressor, named XylR, which is capable of preventing transcription in the absence of xylose in the medium.

The CA_C2613 gene is located upstream of the xylB gene within the main xylose assimilation operon. This organization is very similar to that observed in species bearing a xylose repressor (Gu et al. 2010).

The CA_C3673 gene has an XBS at its promoter, suggesting autoregulation of its transcription. The product of this gene is also the repressor having the greatest identity with XylR homologues belonging to closely related solventogenic Clostridium, such as C. beijerinckii NCIMB 8052.

Most of the genes involved in arabinose assimilation are located within the CA_C1339-CA_C1349 locus. This locus comprises the genes encoding the putative arabinose transporters AraE1 and XylT, the L-arabinose isomerases AraA1 (CA_C1342) and AraA2 (CA_C1346), the ribulokinase AraK (CA_C1344) and the L-ribulose-5-phosphate 4-epimerase AraD (CA_C1341).

A second operon contains the CA_C1529 and CA_C1530 genes encoding the arabinofuranosidase Arb43 and the arabinoside transporter AraT.

All these genes, and also the gene encoding the Xfp phosphoketolase, are placed under the control of the AraR transcriptional repressor (CA_C1340), which is active in the absence of arabinose (Zhang et al. 2012).

A distinctive feature recently highlighted in C. acetobutylicum is the phenotypic difference that exists between growth on xylose and that on arabinose, although the metabolic pathways allowing assimilation of these sugars have many intermediates in common (Servinsky et al. 2010). Specifically, whereas the generation time on arabinose is 78 min, it reaches 287 min on xylose (Servinsky et al. 2012). While xylose permease and xylulokinase activities have been implicated, this difference can also be explained by the action of transcriptional regulatory factors.

Transcriptomic analysis of C. acetobutylicum ATCC 824 cultures in the presence of glucose, xylose or arabinose reveals that the pentose phosphate pathway is similarly induced by xylose and arabinose (Servinsky et al. 2010). In contrast, the expression of the phosphoketolase Xfp has a different expression profile. Specifically, xfp is weakly induced by xylose (by a factor of 3 compared with glucose) but strongly induced by arabinose (by a factor of 185 compared with glucose) (Servinsky et al. 2012). The pronounced contrast in induction of xfp gene expression in the absence of arabinose may be explained by the presence of an AraR-binding-site upstream of this gene. Thus, one of the most noteworthy consequences of transcriptomic analysis of a mutant whose araR gene has been inactivated is the enhancement of xfp gene transcription by a factor of greater than 1000 (Zhang et al. 2012). This transcriptional regulation of phosphoketolase helps to partially hinder xylose assimilation in the absence of arabinose.

Recent work has also shown that, in addition to being partially repressed in the absence of arabinose, xylose assimilation is reduced in the presence of arabinose. Specifically, cultures performed in the presence of xylose and arabinose show minimal xylose consumption, whereas the arabinose assimilation level does not differ from the level that may be observed on arabinose alone (Aristilde et al. 2014). An analysis of metabolic flows confirms the preferential assimilation of arabinose relative to cultured xylose on these two sugars (Aristilde et al. 2014). Servinsky et al. also showed that the preference for arabinose over xylose cannot be entirely explained by the inherent growth rates of these two carbohydrates. They thus highlighted the fact that arabinose would be capable of specifically inhibiting the expression of assimilation genes for other sugars, including those for xylose.

The inventors show for the first time that joint inactivation of the genes encoding the AraR and XylR repressors affords a strain which has astonishing coassimilation capabilities, since it is capable of simultaneously assimilating and/or metabolizing two different carbon sources such as arabinose and glucose, without lifting the catabolic repression on xylose assimilation.

One subject described by the inventors is thus a genetically modified bacterium belonging to the genus Clostridium, in particular a Clostridium species of industrial interest, in particular C. acetobutylicum, characterized in that it does not express the xylR and/or araR gene products, or in that it expresses non-functional versions thereof.

In the present context, the term “xylR gene” refers in particular to the sequence SEQ ID NO: 1 (CA_C2613) or the sequence SEQ ID NO: 2 (CA_C3673). The term “xylR gene” also refers to variants of said sequences SEQ ID NO: 1 and SEQ ID NO: 2, in particular variants having sequence homology with one of said sequences SEQ ID NO: 1 or SEQ ID NO: 2.

In the context of the present invention, the term “araR gene” refers in particular to the sequence SEQ ID NO: 3 (CA_C1340) or a variant of said sequence, in particular a variant presenting sequence homology with said sequence SEQ ID NO: 3.

A typical example of a variant according to the invention has sequence homology with the sequence SEQ ID NO: 1, with SEQ ID NO: 2 or with the sequence SEQ ID NO: 3 of between 95% and 100%, and preferably between 96% and 100%. The sequence SEQ ID NO: 1, SEQ ID NO: 2 or SEQ ID NO: 3 and its variant are, for example, at least 95% homologous, for example at least 96% homologous, at least 97% homologous, at least 98% homologous or at least 99% homologous. According to a preferred embodiment, the sequence SEQ ID NO: 1, SEQ ID NO: 2 or SEQ ID NO: 3 and its variant have sequences which are at least 96%, at least 97% or at least 98% homologous.

The term “xylR gene” also refers to a sequence encoding a functional fragment or variant of the XylR protein, in particular a protein that is able to/capable of repressing, reducing or preventing (or that is involved in the repression of), for example in the absence of xylose, the expression of an enzyme of the xylose assimilation pathway, for example that is able to/capable of repressing, reducing or preventing the transcription of at least one gene chosen from the gene encoding xylose isomerase “XylA” (encoded by gene CA_C2610, SEQ ID NO: 4), the gene encoding xylulokinase “XylB” (encoded by gene CA_C2612, SEQ ID NO: 5), a gene encoding a permease involved in xylose import, for instance the protein “AraE1” (encoded by gene CA_C1339, SEQ ID NO: 6), the “XylT” protein (encoded by gene CA_C1345, SEQ ID NO: 7), or the “XynT” protein (encoded by gene CA_C3451, SEQ ID NO: 8), and the gene encoding the 1,4-β-D-xylosidase “XynB” (encoded by gene CA_C3452, SEQ ID NO: 9).

The term “xylR gene” also refers to a variant nucleotide sequence encoding a protein that is capable of binding, in particular, the promoter (SEQ ID NO: 10) of the “xylA” gene (CA_C2610, SEQ ID NO: 4), the promoter (SEQ ID NO: 11) of the “xylB” gene (CA_C2612, SEQ ID NO: 5) or an XBS (XylR-binding site) operator sequence chosen from SEQ ID NO: 12 and SEQ ID NO: 13, for example. The protein encoded by the variant nucleotide sequence is preferably capable of recognizing an XBS (XyIR-Binding site) sequence such as, for example, SEQ ID NO: 12 or SEQ ID NO: 13, and/or of repressing, reducing or preventing the expression of said genes.

In a particular embodiment, the term “xylR gene” also refers to a sequence encoding a functional fragment or variant of the XylR protein, in particular a protein that is able to/capable of repressing, reducing or preventing (or that is involved in the repression of), for example in the presence of xylose, the expression of an enzyme of the xylose assimilation pathway as described above.

The term “araR gene” also refers to a sequence encoding a functional fragment or variant of the AraR protein, in particular a protein that is able to/capable of repressing, reducing or preventing (or that is involved in the repression of), for example in the absence of arabinose, the expression of an enzyme of the xylose assimilation pathway or the arabinose assimilation pathway, in particular that is capable of partially preventing the assimilation of xylose or arabinose.

The term “araR gene” also refers to a sequence that is able to/capable of repressing, reducing or preventing, for example in the absence of arabinose, the transcription of at least one gene chosen from a gene encoding a transporter, or presumed transporter, of arabinose “XylA”, such as “AraE1” (encoded by gene CA_C1339, SEQ ID NO: 6) or “XylT” (encoded by gene CA_C1345, SEQ ID NO: 7); a gene encoding an L-arabinose isomerase such as “AraA1” (encoded by gene CA_C1342, SEQ ID NO: 14) or “AraA2” (encoded by gene CA_C1346, SEQ ID NO: 15); the gene encoding ribulokinase “AraK” (encoded by gene CA_C1344, SEQ ID NO: 16); the gene encoding L-ribulose-5-phosphate 4-epimerase “AraD” (encoded by gene CA_C1341, SEQ ID NO: 17); the gene encoding the arabinofuranosidase “Arb43” (encoded by gene CA_C1529, SEQ ID NO: 18); the gene encoding the arabinoside transporter “AraT” (encoded by gene CA_C1530, SEQ ID NO: 19); and the xfp gene (CA_C1343, SEQ ID NO: 20) encoding a phosphoketolase. The term “araR gene” also refers to a variant nucleotide sequence encoding a protein that is capable of binding, in particular, the promoter of an “ara” gene such as “araE1”, “araA1”, “araA2”, “araK” or “araD”, for example a sequence chosen from SEQ ID NO: 21, SEQ ID NO: 22, SEQ ID NO: 23, and SEQ ID NO: 24, or an “ABS” (AraR-binding site) operator sequence chosen for example from SEQ ID NO: 25, SEQ ID NO: 26, and SEQ ID NO: 27. The protein encoded by the variant nucleotide sequence is preferably capable of recognizing an ABS (AraR-binding site) sequence, for instance SEQ ID NO: 25 or SEQ ID NO: 26, and/or of repressing, reducing or preventing the expression of one or more of said genes encoding an enzyme involved in the arabinose assimilation pathway.

In a particular embodiment, The term “araR gene” also refers to a sequence encoding a functional fragment or variant of the AraR protein, in particular a protein that is able to/capable of repressing, reducing or preventing (or that is involved in the repression of), for example in the presence of arabinose, the expression of an enzyme as described above of the xylose assimilation pathway or the arabinose assimilation pathway.

When the present text refers to a genetically modified bacterium belonging to the genus Clostridium, characterized in that it does not express the xylR and/or araR gene products, or expresses non-functional versions thereof, the expression “non-functional version of the xylR, or araR, gene product” denotes a protein, typically a protein identified in the present text as “XylR” or “AraR”, which is non-functional, i.e. which is incapable of exercising the function of the protein encoded by the wild-type version of the gene in question. In the case of the product of the xylR gene, the non-functional version of said product denotes a protein that is incapable of repressing, reducing or preventing the expression of an enzyme of the xylose and/or arabinose assimilation pathway, for example that is incapable of repressing, reducing or preventing the transcription of at least one gene chosen from the gene encoding xylose isomerase “XylA”, the gene encoding xylulokinase “XylB”, a gene encoding a permease involved in xylose import, for instance the protein “AraE1”, the protein “XylT”, or the protein “XynT”, and the gene encoding 1,4-β-D-xylosidase “XynB”.

In the case of the araR gene product, the non-functional version of said product denotes a protein that is incapable of repressing, reducing or preventing the expression of an enzyme of the xylose assimilation pathway and/or the arabinose assimilation pathway, in particular that is capable of partially preventing xylose assimilation. This protein is thus incapable of repressing, reducing or preventing the transcription of at least one gene chosen from a gene encoding an arabinose “XylA” transporter, or presumed transporter, such as “AraE1” or “XylT”; a gene encoding an L-arabinose isomerase such as “AraA1” or “AraA2”; the gene encoding the ribulokinase “AraK”; the gene encoding the L-ribulose-5-phosphate 4-epimerase “AraD”; the gene encoding the arabinofuranosidase “Arb43”; the gene encoding the arabinoside transporter “AraT”; and the xfp gene encoding a phosphoketolase.

The term “bacteria of the Clostridium genus” means in particular Clostridium species said to be of industrial interest, typically solventogenic or acetogenic bacteria of the Clostridium genus. The term “bacteria of the Clostridium genus” includes wild-type bacteria and also strains derived therefrom, genetically modified to improve their performance.

The term “Clostridium species of industrial interest” or “Clostridium bacteria of industrial interest” means species that are capable of producing, by fermentation, solvents such as ethanol, butanol, acetone or isopropanol and/or acids such as butyric acid or acetic acid, from sugars or oses, typically from sugars comprising 5 carbon atoms such as xylose, arabinose or fructose, from sugars comprising 6 carbon atoms such as glucose or mannose, from polysaccharides or polyosides such as cellulose or hemicellulose, and/or from any other source of carbon that can be assimilated and used by bacteria of the Clostridium genus (for example CO, CO₂ and methanol). Examples of solventogenic bacteria of interest are bacteria of the Clostridium genus that produce acetone, butanol, ethanol and/or isopropanol, such as the strains identified in the literature as the “ABE strain” [strains performing fermentations allowing the production of acetone, butanol and ethanol], “IBE strain” [strains performing fermentations allowing the production of isopropanol (by reduction of acetone), butanol and ethanol] and “AIBE strain” [strains performing fermentations allowing the production of acetone, isopropanol, butanol and ethanol]. Solventogenic bacteria of the Clostridium genus may be selected from, for example, but not limited to, C. acetobutylicum, C. cellulolyticum, C. phytofermentans, C. beijerinckii, C. saccharobutylicum, C. saccharoperbutylacetonicum, C. sporogenes, C. butyricum, C. aurantibutyricum and C. tyrobutyricum, preferably from C. acetobutylicum, C. beijerinckii, C. butyricum, C. tyrobutyricum and C. cellulolyticum, and even more preferably from C. acetobutylicum and C. beijerinckii. A most preferred solventogenic bacterium is C. acetobutylicum.

The acetogenic bacteria of interest are bacteria that produce acids and/or solvents from CO₂ and H₂. Acetogenic bacteria of the Clostridium genus may be selected, for example, from C. aceticum, C. thermoaceticum, C. ljungdahlii, C. autoethanogenum, C. difficile, C. scatologenes and C. carboxidivorans. In a particular embodiment, the bacterium of the genus Clostridium concerned is an “ABE strain”, preferably of the strain C. acetobutylicum, for example C. acetobutylicum strain DSM 792 (also known as ATCC 824 or LMG 5710).

In another particular embodiment, the bacterium of the genus Clostridium concerned is an “ABE strain”, preferably the C. beijerinckii bacterium, for example the NCIMB 8052 strain of C. beijerinckii. In another particular embodiment, the bacterium of the genus Clostridium concerned is an “IBE strain”, preferably a subclade of C. beijerinckii selected from DSM 6423, LMG 7814, LMG 7815, NRRL B-593 and NCCB 27006.

Thus, according to a preferred embodiment, the bacterium according to the invention belonging to the genus Clostridium is a Clostridium bacterium of industrial interest, in particular a solventogenic bacterium, which is capable, in the wild-type state, of producing solvents and/or acids by fermentation from a carbon source, said carbon source being chosen from, for example, a sugar, CO, CO₂, an alcohol, and an organic acid. In a particular preferred embodiment, the carbon source is a sugar, in particular a sugar comprising 5 carbon atoms such as xylose or arabinose, a sugar comprising 6 carbon atoms such as glucose, fructose or mannose, or a polysaccharide or polyoside such as cellulose or hemicellulose.

A particularly preferred bacterium according to the invention is a Clostridium bacterium that is capable of simultaneously fermenting at least one hexose and one pentose. A particularly preferred bacterium according to the invention is a Clostridium bacterium that is capable of simultaneously fermenting the carbon-based elements of a substrate comprising at least two carbon-based elements chosen from glucose, arabinose, xylose, mannose and galactose, preferably chosen from glucose, arabinose and xylose.

A person skilled in the art can thus readily verify whether or not said Clostridium bacterium is a bacterium according to the invention, by culturing said bacterium on a substrate comprising several carbon sources (for example according to the method taught by Grimmler et al., 2010): if the bacterium ferments/assimilates at least two different carbon sources simultaneously (versus consecutively, i.e. one after the other), it is a bacterium according to the invention. Thus, a wild-type Clostridium bacterium in the presence of a substrate comprising glucose, arabinose and xylose, ferments first glucose, then arabinose and then xylose. On the contrary, a bacterium according to the invention is capable of simultaneously fermenting/assimilating (i.e. at the same time) at least two of the three sugars present in the substrate, namely glucose and arabinose in the example provided (xylose being assimilated in a second stage).

Another simple manner of readily verifying whether or not the Clostridium bacterium is a bacterium according to the invention consists in measuring the bacterium's growth rate. The observation of faster growth for this bacterium than in a bacterium expressing a functional AraR protein, or a mixture of functional XylR and AraR proteins, on a medium comprising xylose as the sole carbon source, will be regarded as an indication that the bacterium has been obtained by means of a process according to the invention (comprising the deletion or inactivation of the xylR and araR genes so as to prevent or reduce the expression of functional XylR and AraR proteins). In practice, a person skilled in the art considers that a wild-type Clostridium bacterium has a maximum growth rate of between about 0.10 and about 0.25; preferentially about 0.15; on a medium comprising xylose as the sole carbon source (Servinsky et al., 2010). The inventors have observed that a bacterium modified according to the invention has a maximum growth rate of between about 0.45 and about 0.60; preferentially greater than about 0.50, on such a medium. Another method for verifying whether or not Clostridium is a bacterium according to the invention consists in sequencing the CA_C1340, CA_C2613 and CA_C3673 loci and checking whether or not they have been modified relative to their wild-type version, with a view to preventing or reducing the expression of functional XylR and AraR proteins.

A particularly preferred bacterium belongs to the species C. acetobutylicum and, as explained above, does not express the gene products of sequences SEQ ID NO: 1 (CA_C2613) or a sequence at least 95%, for example at least 96%, at least 97% or at least 98% homologous therewith, of SEQ ID NO: 2 (CA_C3673) or a sequence homologous to at least 95%, for example at least 96%, at least 97% or at least 98% therewith, and of SEQ ID NO: 3 (CA_C1340) or a sequence homologous to at least 95%, for example at least 96%, at least 97% or at least 98% therewith, or expresses non-functional versions of said expression products. This particularly preferred bacterium has the technical capabilities described above, i.e. it is capable of fermenting/assimilating at least two different carbon sources simultaneously, and grows faster than a bacterium expressing a functional AraR protein, or a mixture of the functional XylR and AraR proteins, on a medium comprising xylose as the sole carbon source.

Such a strain is characterized for the first time in the context of the present patent application.

This strain was registered on 13 Jun. 2022 under deposit number LMG P-32703 at the BCCM-LMG collection. In this strain, the xylR and araR genes have been (jointly) inactivated. The description also relates to any derived bacterium, clone, mutant or genetically modified version thereof, typically free of the xylR and araR genes, or in which said genes have also been inactivated.

Another subject of the invention is the use of a bacterium according to the invention for simultaneously fermenting two different sources of carbon, for example at least one hexose and one pentose, or even the carbon elements of a substrate comprising at least two carbon elements chosen from glucose, arabinose, xylose, mannose and galactose. In a particular embodiment, the bacterium according to the invention is for example capable of simultaneously fermenting glucose and arabinose, glucose and xylose, glucose and mannose, glucose and galactose, arabinose and xylose, arabinose and mannose, arabinose and galactose, xylose and mannose, xylose and galactose or mannose and galactose.

In a particular preferred embodiment, the bacterium according to the invention is capable of fermenting a substrate comprising at least two sugars chosen from glucose, arabinose and xylose, for example glucose and arabinose, glucose and xylose, or arabinose and xylose.

The genetically modified bacterium according to the invention may be advantageously used to produce a solvent, for example a biofuel, or a mixture of solvents, for example a biofuel mixture, in particular on an industrial scale.

The invention is also directed towards a fermentation process, typically an industrial process, involving the use of a bacterium according to the invention.

By virtue of the present invention, the use of second-generation (“2G”) substrates, notably of lignocellulose, is now greatly facilitated. The present invention offers a welcome solution that will allow tensions between the energy and food sectors to be reduced.

A genetically modified bacterium according to the invention may also be advantageously used to produce a sugar or a biobased molecule from biomass, in particular lignocellulosic biomass, or from a dedicated energy crop.

In the context of the present invention, the term “biobased molecule” means a molecule whose specificity is that the starting material used for its production is necessarily derived from plant biomass, preferably lignocellulosic biomass, and not from resources of fossil origin such as oil, coal or natural gas.

Non-limiting examples of lignocellulosic biomasses include, for example, agricultural residues (notably straw waste, corn cobs, sugarcane bagasse, etc.), but also forestry residues, sawmill residues (typically wood chips) or any other type of ligneous residue.

Cellulosic biomass may also be the product of a particular dedicated crop such as an energy crop, dedicated for example to the cultivation of annual plants (“whole plant” use, for example, of wheat or corn). It may otherwise be, for example, a crop of perennial forage plants (for example fescue, cocksfoot, ryegrass), a crop of herbaceous plants (for example miscanthus or switchgrass), or a shrub crop (short or very short rotation coppice, for example willow, poplar, chestnut or eucalyptus).

Another biomass that may be used in the context of the invention may be a 1G substrate derived from sugar-yielding plants (for instance sugar cane or sugar beet), cereal plants (for instance corn or wheat) and/or oil-yielding plants (for instance soybean, rapeseed or palm).

The invention is moreover directed towards a process for producing a recombinant bacterium according to the invention, comprising the deletion or inactivation of the xylR and araR genes so as to prevent or reduce the expression of functional XylR and AraR proteins.

The known modification processes with the best performance for obtaining genetically modified strains are based on homologous recombination events, which allow the genome to be modified in a precise and stable manner.

A particular production process according to the invention comprises a step of transforming the bacterium by introducing a nucleic acid of interest into said bacterium.

For the purposes of the invention, the term “nucleic acid” means any natural, synthetic, semi-synthetic or recombinant DNA or RNA molecule, possibly chemically modified (i.e. comprising non-natural bases, modified nucleotides including, for example, a modified bond, modified bases and/or modified sugars), or optimized so that the codons of the transcripts synthesized from the coding sequences are the codons most frequently found in a bacterium of the genus Clostridium with a view to its use therein. In the case of the Clostridium genus, the optimized codons are typically codons rich in adenine (“A”) and thymine (“T”) bases.

In the peptide sequences described in the present document, the amino acids are represented by their one-letter code according to the following nomenclature: C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan and Y: tyrosine.

In a particular embodiment described, the nucleic acid of interest comprises at least two regions each complementary to a target sequence, which is at least 100% identical or at least 80% identical, preferably at least 85%, 90%, 95%, 96%, 97%, 98% or 99% identical to said targeted DNA region/portion/sequence within the bacterial genome. These regions are capable of hybridizing to all or part of the sequence complementary to said region/portion/sequence, typically to a sequence as described above comprising at least 1 nucleotide, preferably at least 100 nucleotides, typically between 100 and 1000 nucleotides. The complementary regions of the target sequence present within the nucleic acid of interest may recognize, and preferably target, the 5′ and 3′ flanking regions of the targeted sequence in a genetic modification tool known to those skilled in the art, typically any tool based on homologous recombination.

In a particular preferred embodiment, a portion of the nucleic acid of interest also recognizes (binds at least in part to), and preferably targets, i.e. recognizes and allows the cleavage of, in the genome of a Clostridium bacterium of interest, at least one strand of i) a target sequence, ii) a sequence controlling the transcription of a target sequence, or iii) a sequence flanking a target sequence. The recognized sequence is also identified in the present text as a “target sequence” or “targeted sequence”.

In this same preferred particular embodiment, the nucleic acid of interest comprises at least one region complementary to the target sequence which is at least 100% identical or at least 80% identical, preferably at least 85%, 90%, 95%, 96%, 97%, 98% or 99% identical to the targeted DNA region/portion/sequence within the bacterial genome, and is capable of hybridizing to all or part of the sequence complementary to said region/portion/sequence, typically to a sequence comprising at least 5 nucleotides, preferably at least 5, 10, 14, 15, 20, 25, 30, 35 or 40 nucleotides, typically between 15 and 30 nucleotides, preferably to a sequence comprising 18, 19, 20, 21, 22, 23, 24 or 25 nucleotides.

The nucleic acid(s) of interest as described in the context of the present invention are capable of deleting said target sequence(s) from the bacterial genome or modifying their expression, for instance modulating/regulating them, in particular inhibiting them, preferably modifying them so as to render said bacterium incapable of expressing one or more proteins, in particular one or more functional proteins, from said sequence(s).

The “nucleic acids of interest”, typically expression cassettes or vectors, may be constructed via conventional techniques well known to those skilled in the art, and may include one or more promoters, bacterial origins of replication (ORI sequences), termination sequences, selection genes, for example antibiotic resistance genes, and sequences (“flanking regions”) enabling targeted insertion of the cassette or vector. ORI sequences of interest may, for example, be chosen from pIP404, pAMB1, repH (origin of replication in C. acetobutylicum), ColE1 or rep (origin of replication in E. coli), or any other origin of replication enabling the vector, typically the plasmid, to be maintained within a bacterial cell belonging to the genus Clostridium. Termination sequences of interest may be chosen, for example, from those of the ad and thl genes, the bcs operon, or any other terminator, well known to those skilled in the art, enabling transcription to be stopped within a bacterial cell belonging to the genus Clostridium. Selection genes (resistance genes) of interest may be chosen from ermB, catP, bla, tetA, tetM, and/or any other gene for resistance to ampicillin, erythromycin, chloramphenicol, thiamphenicol, spectinomycin, tetracycline, or any other antibiotic, well known to those skilled in the art, which may be used to select bacteria of the Clostridium genus.

The nucleic acid of interest may be a natural or synthetic RNA, or produced via a recombinant technique. This nucleic acid of interest may be prepared via any method known to those skilled in the art, for instance chemical synthesis, in vivo transcription or amplification techniques. When the nucleic acid(s) of interest are introduced into the cell directly in the form of RNA molecules (mature or precursor), for example guide RNA (gRNA), these molecules may contain modified nucleotides or chemical modifications allowing them, for example, to increase their resistance to nucleases and thus increase their lifespan in the cell. They may notably comprise at least one modified or unnatural nucleotide, for instance a nucleotide comprising a modified base, such as inosine, methyl-5-deoxycytidine, dimethylamino-5-deoxyuridine, deoxyuridine, diamino-2,6-purine, bromo-5-deoxyuridine or any other modified base allowing hybridization.

The nucleic acids of interest used according to the invention may also be modified at the internucleotide bond level, for instance like phosphorothioates, H-phosphonates or alkyl-phosphonates, or at the backbone level, for instance like alpha-oligonucleotides, 2′-O-alkyl riboses or PNAs (Peptide Nucleic Acids) (Egholm et al., 1992).

In the context of the present description, a particular example of a nucleic acid of interest, used for transforming and/or genetically modifying a bacterium of interest, is a DNA fragment i) recognizing a coding sequence, ii) controlling transcription of a coding sequence, or iii) flanking a coding sequence, the XylR repressor or the AraR repressor.

In a preferred embodiment, the nucleic acid of interest is selected from plasmid pGRNA-ΔCA_C1340 of sequence SEQ ID NO: 28, plasmid pGRNA-ΔCA_C2613 of sequence SEQ ID NO: 29 or plasmid pGRNA-ΔCA_C3673 of sequence SEQ ID NO: 30.

A nucleic acid of particular interest described by the inventors is for example a vector, preferably a plasmid, for example the plasmid pGRNA-ΔCA_C1340 of sequence SEQ ID NO: 28, the plasmid pGRNA-ΔCA_C2613 of sequence SEQ ID NO: 29 or the plasmid pGRNA-ΔCA_C3673 of sequence SEQ ID NO: 30, described in the experimental section of the present description.

The or one of the recognized sequences (target sequence(s)) is preferably one of the sequences SEQ ID NO: 1, SEQ ID NO: 2 or SEQ ID NO: 3 corresponding to the xylR and araR genes encoding an XyIR protein and the AraR protein respectively, or an amino acid sequence at least 70%, 75%, 80%, 85%, 90% or 95% identical to said protein, or a sequence comprising all or at least 95%, 96%, 97%, 98% or 99% of the sequence SEQ ID NO: 1, SEQ ID NO: 2 or SEQ ID NO: 3. In other words, the recognized sequence may be a sequence comprising at least 1 nucleotide, preferably at least 1, 2, 3, 4, 5, 10, 15, 20, 25, 30, 35 or 40 nucleotides, typically between 1 and 40 nucleotides, preferably a sequence comprising 14, 15, 16, 17, 18, 19, 20, 21, 22, 23, 24, 25, 26, 27, 28, 29 or 30 nucleotides of the sequence SEQ ID NO: 1, SEQ ID NO: 2 or SEQ ID NO: 3.

According to another particular example, the target sequence may also be a sequence controlling the transcription of a coding sequence as described previously, typically a promoter sequence, for example the promoter sequence (SEQ ID NO: 10) of the “XylA” gene (CA_C2610, SEQ ID NO: 4), or that (SEQ ID NO: 11) of the “XylB” gene (CA_C2612, SEQ ID NO: 5). The nucleic acid of interest then recognizes, and is thus typically capable of binding to, a sequence controlling the transcription of a coding sequence as described previously.

According to another particular example, the target sequence may be a sequence flanking a coding sequence as described previously, for example a sequence flanking the sequence SEQ ID NO: 1, SEQ ID NO: 2 or SEQ ID NO: 3, or a sequence at least 70% identical thereto. Such a flanking sequence typically comprises 1, 10 or 20 and 1000 nucleotides, for example between 1, 10 or 20 and 900, 800, 700, 600, 500, 400, 300 or 200 nucleotides, between 1, 10 or 20 and 100 nucleotides, between 1, 10 or 20 and 50 nucleotides, or between 1, 10 or 20 and 40 nucleotides, for example between 10 and 40 nucleotides, between 10 and 30 nucleotides, between 10 and 20 nucleotides, between 20 and 30 nucleotides, between 15 and 40 nucleotides, between 15 and 30 nucleotides or between 15 and 20 nucleotides.

According to a particular aspect, the target sequence corresponds to the pair of sequences flanking such a coding sequence, each flanking sequence typically comprising at least 20 nucleotides, typically between 100 and 1000 nucleotides, preferably between 200 and 800 nucleotides.

Preferably, the process according to the invention for producing a recombinant bacterium involves transforming the bacterial cell with at least one nucleic acid of interest, for example two or three nucleic acids of interest as described previously, said nucleic acid(s) of interest being capable i) of recognizing (and capable of at least partly binding) a sequence encoding, controlling the transcription of a sequence encoding, or flanking a sequence encoding the XylR repressor, and ii) recognizing (and capable of at least partly binding) a sequence encoding, controlling the transcription of a sequence encoding, or flanking a sequence encoding the AraR repressor.

As explained above, the sequence encoding, controlling the transcription of a sequence encoding, or flanking a sequence encoding the XylR repressor or the AraR repressor is also identified in the present text as a “target sequence” or “targeted sequence”.

The nucleic acid of interest as described in the context of the present invention is preferably capable of deleting said target sequence from the bacterial genome or modifying its expression, for example modulating/regulating it, in particular inhibiting it, preferably modifying it in such a way as to render said bacterium incapable of expressing a protein (typically an AraR or XylR protein), in particular a functional protein, from said sequence. In a particularly preferred embodiment, the nucleic acid of interest is capable of modifying the bacterium so as to render it incapable of expressing one and/or the other of the AraR and XylR proteins.

The introduction into the bacterium of any nucleic acid(s) of interest may be performed by any direct or indirect method known to those skilled in the art, for example by transformation, conjugation, microinjection, transfection, electroporation, etc., preferably by electroporation (Mermelstein et al, 1993). Moreover, these nucleic acids of interest (for example, DNA fragments, RNA fragments, expression cassettes or expression vectors) may be incorporated into the bacterial genome via techniques that are also well known to those skilled in the art.

In a particular embodiment, the process for transforming, and preferably genetically modifying, a bacterium as described herein comprises a step of transforming the bacterium by introducing into said bacterium a nucleic acid of interest according to the invention as described above and involves a genetic modification tool, for example a genetic modification tool selected from a CRISPR tool, an insertional mutagenesis tool, for example based on the use of type II introns (for example the Targetron® tool or the ClosTron® tool) and an allelic exchange tool (for example the ACER tool).

The process for transforming, and preferably also genetically modifying, a Clostridium bacterium may also comprise a step of obtaining, recovering, selecting or isolating the transformed bacterium, i.e. the bacterium exhibiting the desired recombination(s)/modification(s)/optimization(s).

In a particular embodiment, the process according to the invention is based on the use of (implements) the CRISPR (Clustered Regularly Interspaced Short Palindromic Repeats) technology/genetic tool, in particular the CRISPR/Cas (CRISPR-associated protein) genetic tool. The present invention may be performed using a conventional CRISPR/Cas genetic tool using a single plasmid comprising a nuclease, a gRNA and a repair template as described by Wang et al. (2015). A person skilled in the art can readily define the sequence and structure of the gRNAs according to the chromosomal region or mobile genetic element to be targeted using well-known techniques (see for example the article by DiCarlo et al., 2013). The inventors have otherwise developed and described a genetic tool for bacterial modification, suitable for bacteria of the genus Clostridium, which may be used in the context of the present invention, based on the use of two plasmids (cf. WO 2017/064439, Wasels et al., 2017).

In another embodiment, the process according to the invention is based on the use of insertional mutagenesis tools, for example the use of type II introns, and involves, for example, the ClosTron® technology/genetic tool or the Targetron® genetic tool.

Targetron® technology is based on the use of a reprogrammable group II intron (based on Lactococcus lactis intron Ll.ltrB), which is capable of rapidly incorporating the bacterial genome at a desired locus (Chen et al., 2005, Wang et al., 2013), typically with the aim of inactivating a targeted gene. The mechanisms for both the recognition of the edited area and the insertion into the genome by reverse splicing are based on homology between the intron and said area on the one hand, and on the activity of a protein (ltrA) on the other.

ClosTron® technology is based on a similar approach, complemented by the addition of a selection marker into the intron sequence (Heap et al., 2007). This marker allows the integration of the intron into the genome to be selected, and thus facilitates the production of the desired mutants. This genetic system also exploits type I introns. Specifically, the selection marker (called RAM for retrotransposition-activated marker) is interrupted with such a genetic element, which prevents its expression from the plasmid (for a more precise description of the system: Zhong et al.). Splicing of this genetic element occurs prior to integration into the genome, allowing production of a chromosome having an active form of the resistance gene. An optimized version of the system comprises FLP/FRT sites upstream and downstream of this gene, allowing FRT recombinase to be used to eliminate the resistance gene (Heap et al., 2010).

In another embodiment, the process according to the invention is based on the use of an allelic exchange tool, and involves, for example, the ACER technology/genetic tool.

ACE® technology is based on the use of an auxotrophic mutant (for uracil in C. acetobutylicum ATCC 824 by deletion of the pyrE gene, which also causes resistance to 5-fluoroorotic acid (A-5-FO); Heap et al., 2012). The system uses the allelic exchange mechanism, well known to those skilled in the art. Following transformation with a pseudo-suicidal (very low copy) vector, integration of the latter into the bacterial chromosome by a first allelic exchange event may be verified by virtue of the resistance gene initially present on the plasmid. The integration step may be performed in two different ways, either within the pyrE locus or within another locus. In the case of integration at the pyrE locus, the pyrE gene is also placed on the plasmid, but is not expressed (no functional promoter). The second recombination restores a functional pyrE gene, which can then be selected by auxotrophy (minimal medium, containing no uracil). As the non-functional pyrE gene also has a selectable character (sensitivity to A-5-FO), other integrations can then be envisaged on the same model, successively alternating the state of pyrE between functional and non-functional. In the case of integration into another locus, a genomic area allowing expression of the counter-selection marker after recombination is targeted (typically, in an operon after another gene, preferably a highly expressed gene). This second recombination is then selected by auxotrophy (minimum medium containing no uracil).

In the described embodiments based on the use of type II introns, and involving, for example, the ClosTron® technology/genetic tool or the Targetron® genetic tool, or based on the use of an allelic exchange tool, and involving, for example, the ACE® technology/genetic tool, the targeted sequence is typically one of the sequences described in the present text.

The invention moreover relates to a kit for transforming and/or genetically modifying a bacterium of the genus Clostridium comprising at least one nucleic acid of interest as described herein (for example two nucleic acids of interest, typically a DNA fragment, each recognizing a target sequence) for transforming and preferably genetically modifying a bacterium of the genus Clostridium, and optionally one or more selection molecules. A particular kit comprises the elements essential for the operation of a CRISPR tool (typically at least one nucleic acid that may be used as gRNA, one nucleic acid that may be used as a repair template, at least one primer pair, and an inducer enabling expression of a Cas9 or MAD7 type nuclease), the elements essential for the operation of a tool based on the use of type II introns (typically at least one type II intron, at least one primer pair and an inducer allowing the expression of a retrotranscriptase, for example of the LtrA RT or Tel4c RT type), or the elements essential to the operation of an allelic exchange tool (typically at least two nucleic acids that may be used as homologous recombination templates, and at least one primer pair).

The kits according to the invention may also comprise one or more consumables such as a culture medium, at least one competent bacterium of the Clostridium genus (i.e. conditioned for transformation), or an explanatory leaflet.

The invention typically relates to a kit for performing a transformation and/or genetic modification process described herein using a bacterium of the genus Clostridium.

The invention is directed in particular towards the genetically modified bacterium belonging to the genus Clostridium having the essential feature of not expressing the xylR and araR gene products, or of expressing non-functional versions thereof, and also towards any derived bacterium, clone, mutant or genetically modified version thereof, and uses thereof.

The patent application also describes strain IFP 967, registered on 13 Jun. 2022 under deposit number LMG P-32702 at the BCCM-LMG collection, in which the araR gene has been inactivated, and any derived bacteria, clones, mutants or genetically modified versions thereof, typically free of the xylR and araR genes, or in which said genes have also been inactivated.

The patent application is particularly directed towards the use of a genetically modified bacterium belonging to the genus Clostridium, characterized in that it does not express the xylR or araR gene product, or expresses a non-functional version of the xylR or araR gene, for example strain IFP 967, registered on 13 Jun. 2022 under deposit number LMG P-32702 at the BCCM-LMG collection, for preparing a genetically modified bacterium according to the invention not expressing the xylR and araR gene products, or expressing non-functional versions thereof.

The invention moreover relates to a kit for producing a solvent, a sugar or a biobased molecule using a bacterium belonging to the genus Clostridium, comprising i) a genetically modified bacterium belonging to the genus Clostridium according to the invention, for example IPF968, and ii) a medium, typically a storage medium or a culture medium for said bacterium. The kit may also comprise any explanatory leaflet.

The storage or culture medium for the genetically modified bacterium (belonging to the genus Clostridium) according to the invention present in the kit is preferably supplemented with a carbon source consisting of glucose and at least one pentose, preferably i) glucose and ii) arabinose and/or xylose. This medium preferably comprises between 0.1 and 250 g/L, more preferentially between 1 and 100 g/L, of said carbon source (composed of glucose and at least one pentose).

The storage or culture medium for the genetically modified bacterium is preferably an RCM-type culture medium, more preferentially a GAPES-type culture medium, even more preferentially a CGM-type culture medium.

The invention also relates to a particular kit for producing a solvent or biofuel, or a mixture of solvents or biofuels, by means of a bacterium belonging to the genus Clostridium, said kit comprising i) a bacterium (belonging to the genus Clostridium) genetically modified according to the invention, characterized in that it does not express the xylR and araR gene products, or in that it expresses non-functional versions thereof, and ii) a rich culture medium, preferentially an RCM-type culture medium, more preferentially a GAPES-type culture medium, even more preferentially a CGM-type culture medium, containing at least glucose and a pentose, preferentially arabinose and/or xylose.

The invention moreover relates to the possible uses of the process or kit according to the invention for transforming and/or genetically modifying a bacterium of the genus Clostridium, typically a solventogenic bacterium of the genus Clostridium, for example to generate improved variants of said bacterium.

Finally, the invention relates to the possible uses of the process, the kit or a bacterium of the Clostridium genus transformed and preferably genetically modified according to the invention, in particular to allow the production of solvents or biofuels, or mixtures thereof, typically on an industrial scale.

The examples and figures hereinbelow are intended to illustrate the invention more fully without limiting its scope. In particular, these examples show the production and characterization of bacteria according to the invention, in which the inactivation of araR and/or xylR genes is performed according to a particular preferred embodiment by means of a CRISPR-Cas9 tool. Said genes can be inactivated according to other particular embodiments, well known to those skilled in the art, for example based on gene inactivation by homologous recombination or insertional mutagenesis, as explained above.

### FIGURES

FIG. 1 represents the Map of plasmid pBANak.

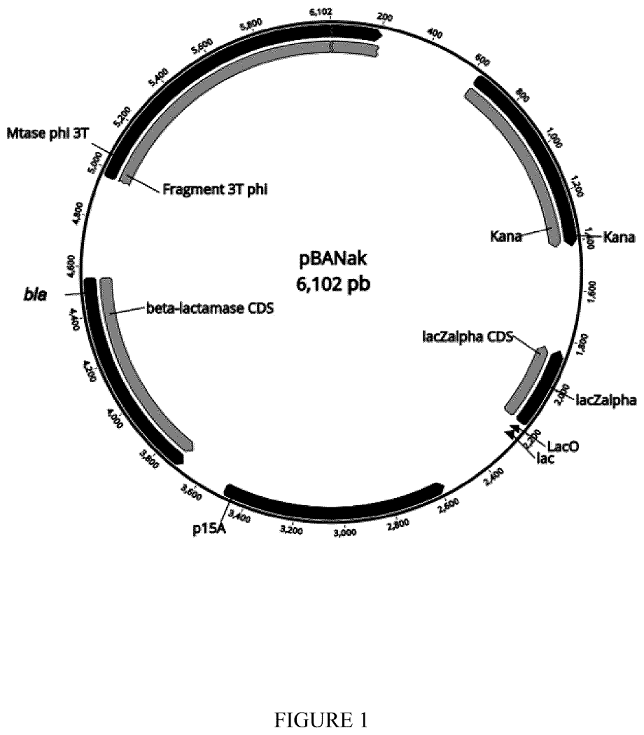

FIG. 2 represents the Map of plasmid pGRNA_ΔCA_C1340

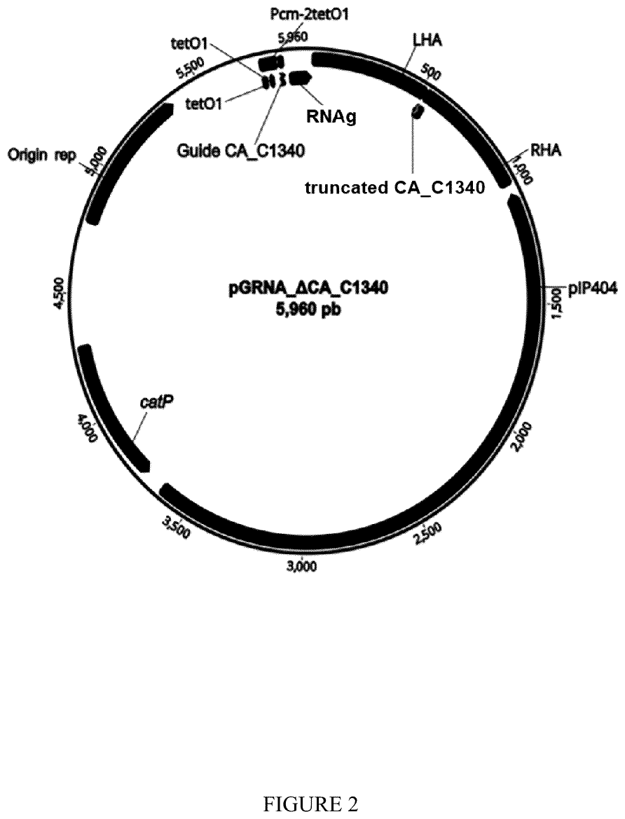

FIG. 3 represents the Map of plasmid pGRNA_ΔCA_C2613

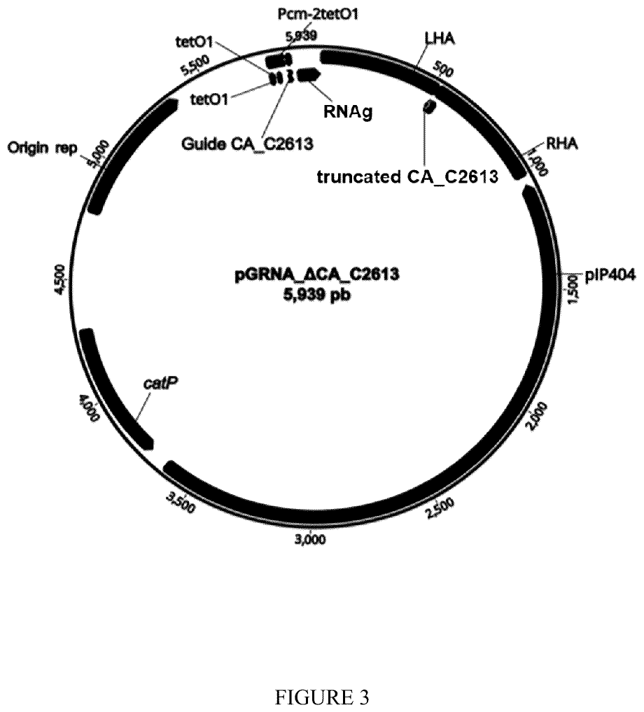

FIG. 4 represents the Map of plasmid pGRNA_ΔCA_C3673

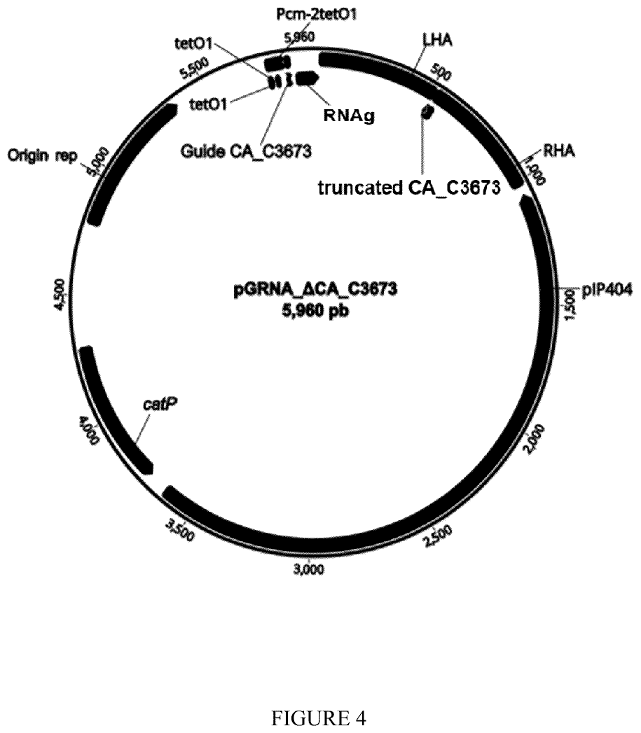

FIG. 5 shows growth kinetics on 30 mM xylose CGM medium.

FIG. 6A represents the consumption kinetics of a mixture of glucose, arabinose and xylose in wild-type C. acetobutylicum strain DSM 792, on CGM medium containing 25 mM glucose, 25 mM arabinose and 70 mM xylose.

FIG. 6B represents the consumption kinetics of a mixture of glucose, arabinose and xylose in C. acetobutylicum strain DSM 792 ΔCA_C2613 ΔCA_C3673, on CGM medium containing 25 mM glucose, 25 mM arabinose and 70 mM xylose.

FIG. 6C represents the consumption kinetics of a mixture of glucose, arabinose and xylose in C. acetobutylicum strain DSM 792 ΔCA_C1340, on CGM medium containing 25 mM glucose, 25 mM arabinose and 70 mM xylose.

FIG. 6D represents the consumption kinetics of a mixture of glucose, arabinose and xylose in C. acetobutylicum strain DSM 792 ΔCA_C1340 ΔCA_C2613 ΔCA_C3673, on CGM medium containing 25 mM glucose, 25 mM arabinose and 70 mM xylose.

### EXAMPLES

### Example 1: C. acetobutylicum Strain ΔCA C2613 ΔCA C3673

### Materials and Methods

### Culture Conditions

C. acetobutylicum DSM 792 was cultured in 2YTG medium (Tryptone 16 g·L⁻¹, yeast extract 10 g·L⁻¹, glucose 5 g·L⁻¹, NaCl 4 g·L⁻¹) and CGM (KH₂PO₄, 0.75 g; K₂HPO₄, 0.75 g; MgSO₄·7H₂O, 0.40 g; MnSO₄·H₂O, 0.01 g; FeSO₄·7H₂O, 0.01 g; NaCl, 1.0 g; asparagine, 2.0 g; yeast extract, 5.0 g; (NH₄)₂SO₄, 2.0 g; carbon source as specified). Escherichia coli NEB 10-beta was grown in LB medium (Tryptone g·L⁻¹, yeast extract 5 g·L⁻¹, NaCl 10 g·L⁻¹). Solid media were produced by adding 15 g·L⁻¹ of agarose to liquid media. Erythromycin (at concentrations of 40 mg·L⁻¹ in 2YTG medium), chloramphenicol (25 or 12.5 mg L⁻¹ in solid or liquid LB medium respectively), thiamphenicol (15 mg L⁻¹ in 2YTG medium) were used as required.

Nucleic acids and plasmid vectors
The list of primers used for all constructs
(name/DNA sequence) is detailed below:

| | |
|---|---|
| CA C2613 LHA Fwd: | AAAAAAGAATTCATTTGTCCACTTATACCAATTCCT (SEQ ID NO: 31) |
| CA_C2613 LHA Rev: | TCCTACTAGATACGGCGGAGTAAAATTAGT (SEQ ID NO: 32) |
| CA C2613 RHA Fwd: | CTCCGCCGTATCTAGTAGGAATCTCCCACT (SEQ ID NO: 33) |
| CA C2613 RHA Rev: | AAAAAAGTCGACAAAGAGAAAATAAGAGGAATACAAAAG (SEQ ID NO: 34) |
| gRNA_CA_C2613 Fwd: | TCATGTTACACTTGGAACAGGCGT (SEQ ID NO: 35) |
| gRNA_CA_C2613_Rev: | AAACACGCCTGTTCCAAGTGTAAC (SEQ ID NO: 36) |
| CA C3673 LHA Fwd: | AAAAAAGGATCCAGAAAGACTTGTTACATCTCTAAGT (SEQ ID NO: 37) |
| CA_C3673_LHA Rev: | TGATATAGACTTAATTGAACAAGATGTAATTTGAAATTAGT (SEQ ID NO: 38) |
| CA C3673 RHA Fwd: | GTTCAATTAAGTCTATATCAAACATATCGCACCAACC (SEQ ID NO: 39) |
| CA C3673 RHA Rev: | AAAAAAGTCGACATGATAATGGGAAATATATAGGAACTTT (SEQ ID NO: 40) |
| gRNA_CA_C3673 Fwd: | TCATGGAGTAGCAAGCTCTACAGG (SEQ ID NO: 41) |
| gRNA_CA_C3673_Rev: | AAACCCTGTAGAGCTTGCTACTCC (SEQ ID NO: 42) |
| CA C2613_verif Fwd: | TCGGCAGCAAAAACTCCAGT (SEQ ID NO: 43) |
| CA_C2613_verif_Rev: | GCAATTGCTGGCGGAATGAA (SEQ ID NO: 44) |
| CA C3673_verif Fwd: | TACTTCATACATATCTAACGCACTCT (SEQ ID NO: 45) |
| CA_C3673_verif Rev: | AAATAACTTCTACTATGAGACAAGCA (SEQ ID NO: 46) |

The following plasmid vectors were constructed:
Plasmid No. 1: pBANak (SEQ ID NO: 47)

It contains the p15A origin of replication, and also the ampR and kanR genes, allowing ampicillin and kanamycin resistance respectively to be conferred. It also contains an expression cassette for Bacillus subtilis phi 3T I methyltransferase, required for methylation of gene-editing vectors prior to transformation in C. acetobutylicum.
Plasmid No. 2: pGRNA_ΔCA_C2613 (SEQ ID NO: 29)

It contains all the elements of the pGRNAind with a guide RNA substitution affording CA_C2613 targeting (Oligo-20-mer obtained by hybridization of the primers gRNA_CA_C2613_Fwd (SEQ ID NO: 35) and gRNA_CA_C2613_Rev (SEQ ID NO: 36) and incorporated into the plasmid via the “golden gate assembly” technique, after digestion with BsaI. It also contains an editing template allowing inactivation of CA_C2613. This editing template was constructed by cloning the LHA and RHA fragments obtained respectively by amplification with the primer pairs CA_C2613_LHA_Fwd (SEQ ID NO: 31)/CA_C2613_LHA Rev (SEQ ID NO: 32) and CA_C2613_RHA_Fwd (SEQ ID NO: 33)/CA_C2613_RHA Rev (SEQ ID NO: 34) from the gDNA of C. acetobutylicum DSM 792. The editing template was cloned between the BamHI and SalI restriction sites.
Plasmid No. 3: pGRNA_ΔCA_C3673 (SEQ ID NO: 30)

It contains all the elements of the pGRNAind with a guide RNA substitution affording CA_C3673 targeting (Oligo-20-mer obtained by hybridization of the primers gRNA_CA_C3673_Fwd (SEQ ID NO: 41) and gRNA_CA_C3673_Rev (SEQ ID NO: 42) and incorporated into the plasmid by the golden gate assembly technique, after digestion with BsaI. It also contains an editing template allowing inactivation of CA_C3673. This editing template was constructed by cloning the LHA and RHA fragments obtained respectively by amplification with the primer pairs CA_C3673_LHA_Fwd (SEQ ID NO: 37)/CA C3673_LHA_Rev (SEQ ID NO: 38) and CA_C3673_RHA_Fwd (SEQ ID NO: 39)/CA_C3673_RHA Rev (SEQ ID NO: 40) from the gDNA of C. acetobutylicum DSM 792. The editing template was cloned between the BamHI and SalI restriction sites.

Transformation of C. acetobutylicum Strains

The plasmids prepared in the NEB 10-beta strain were methylated by transformation into an NEB 10-beta strain also containing the pBANak plasmid. The methylated plasmids thus extracted were then used to transform the C. acetobutylicum strain. Transformation was performed according to the protocol described by Wasels et al. 2020.
Genetic editing of CA_C2613 (SEQ ID NO: 1) and CA_C3673 (SEQ ID NO: 2) genes

Inactivations of the CA_C2613 and CA_C3673 genes in ΔCA_C2613-ΔCA_C3673 strains were performed by gene editing according to the protocol described by Wasels et al. 2020, respectively using the primer pairs CA_C2613_verif_Fwd (SEQ ID NO: 43)/CA_C2613_verif_Rev (SEQ ID NO: 44); CA_C3673_verif_Fwd (SEQ ID NO: 45)/CA_C3673_verif_Rev (SEQ ID NO: 46) for editing validation.

The use of other genetic tools, such as those based on homologous recombination or the insertion of mobile genetic elements, may afford mutants having equivalent genotypes, i.e. no longer expressing the products of the CA_C2613 and CA_C3673 genes, or expressing a non-functional version thereof.

### Results

### Growth Kinetics and Determination of Generation Time

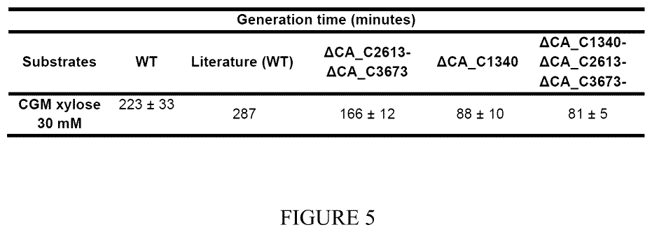

Growth monitoring of wild-type C. acetobutylicum DSM 792 and ΔCA_C2613 ΔCA_C3673 strains was performed in quintuplicate on CGM medium containing 30 mM xylose (FIG. 5).

The results reveal an improvement in generation time when growing on xylose as a carbon source. Also, joint inactivation of the CA_C2613 and CA_C3673 genes allowed a 33% improvement in generation time relative to the wild-type strain.

### Monitoring of Consumption of a Sugar Mixture

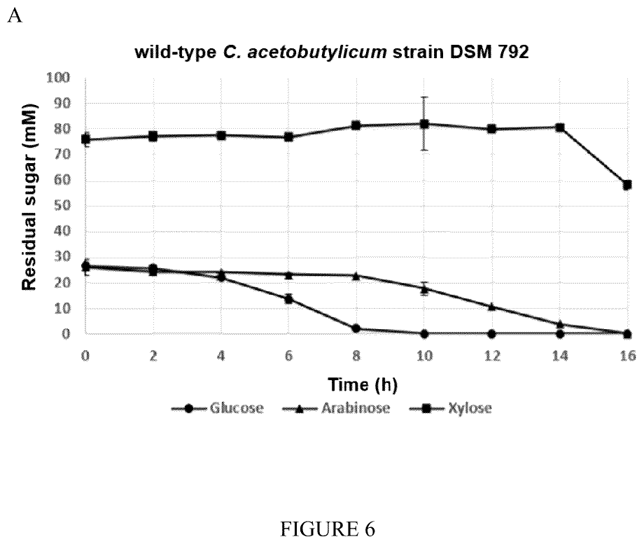

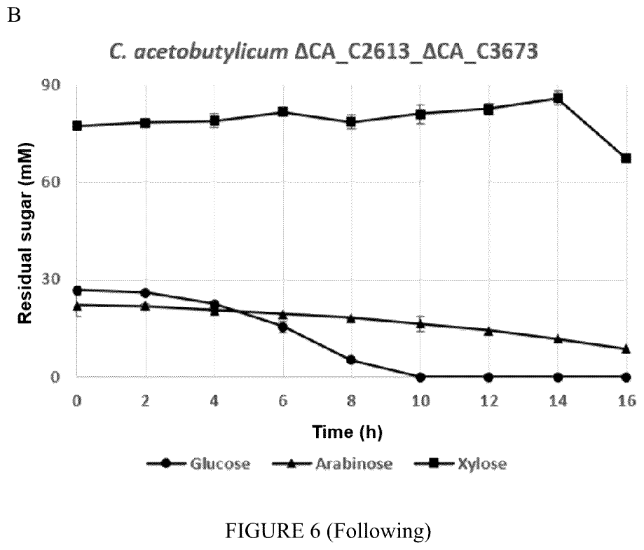

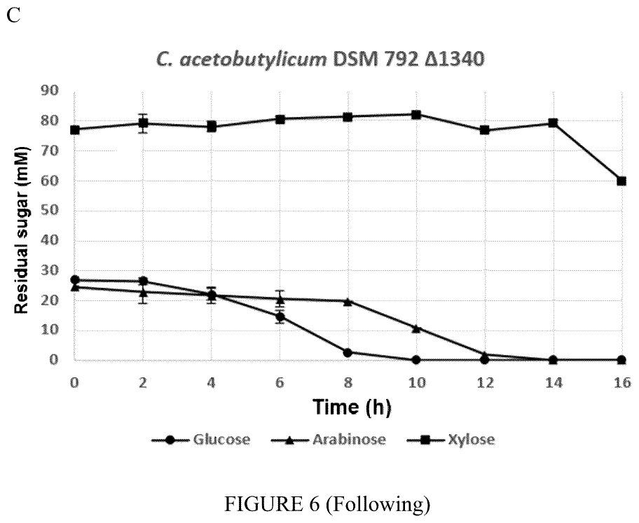

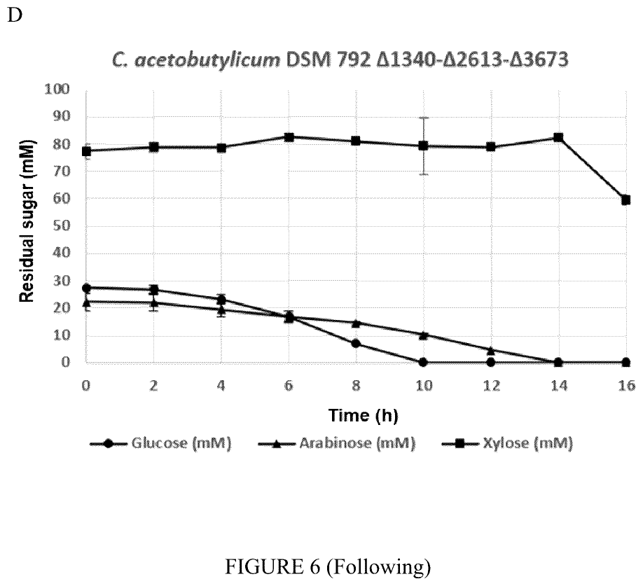

Sugar mixture consumption monitoring was performed in technical triplicates with wild-type C. acetobutylicum DSM 792 and ΔCA_C2613 ΔCA_C3673 strains, on CGM medium containing 25 mM glucose, 25 mM arabinose and 70 mM xylose (FIG. 6).

The results indicate that joint inactivation of CA_C2613 and CA_C3673 shows no significant phenotypic change relative to the wild-type strain.

### Example 2: Strain IFP 967 (LMG P-32702), C. acetobutylicum ΔCA C1340

### Materials and Methods

### Growing Conditions

C. acetobutylicum DSM 792 was grown in 2YTG medium (Tryptone 16 g·L⁻¹, yeast extract 10 g·L⁻¹, glucose 5 g·L⁻¹, NaCl 4 g·L⁻¹) and CGM (KH₂PO₄, 0.75 g; K₂HPO₄, 0.75 g; MgSO_{4·7}H₂O, 0.40 g; MnSO₄·H₂O, 0.01 g; FeSO₄·7H₂O, 0.01 g; NaCl, 1.0 g; asparagine, 2.0 g; yeast extract, 5.0 g; (NH₄)₂SO₄, 2.0 g; carbon source as specified). Escherichia coli NEB 10-beta was grown in LB medium (Tryptone 10 g·L⁻¹, yeast extract 5 g·L⁻¹, NaCl 10 g·L⁻¹). Solid media were produced by adding 15 g·L⁻¹ of agarose to liquid media. Erythromycin (at concentrations of 40 mg·L⁻¹ in 2YTG medium), chloramphenicol (25 or 12.5 mg L⁻¹ in solid or liquid LB medium respectively), thiamphenicol (15 mg L⁻¹ in 2YTG medium) were used as required.

### Nucleic Acids and Plasmid Vectors

The list of primers used for all constructs (name/DNA sequence) is detailed below:

| | |
|---|---|
| CA C1340 LHA Fwd: | AAAAAAGGATCCACCAGAAAAACCAACGCCTAGTA |
| | (SEQ ID NO: 48) |
| CA C1340 LHA Rev: | AGAAGTAGAAGAATTAAAATTGAAATAATTTATTTAGTCCCTTGCC |
| | (SEQ ID NO: 49) |
| CA_C1340_RHA Fwd: | TTTCAATTTTAATTCTTCTACTTCTTCATATTTGTGCT |
| | (SEQ ID NO: 50) |
| CA_C1340_RHA Rev: | AAAAAAGTCGACTAATTACTCATTACATACGTTATTTTTCAGT |
| | (SEQ ID NO: 51) |
| gRNA_CA_C1340 Fwd: | TCATTTTAAGGTCGATGATTCACA |
| | (SEQ ID NO: 52) |
| gRNA_CA_C1340 Rev: | AAACTGTGAATCATCGACCTTAAA |
| | (SEQ ID NO: 53) |
| CA_C1340_verif_Fwd: | CCTACCAATAGCATCGCCCAAGA |
| | (SEQ ID NO: 54) |
| CA_C1340_verif_Rev: | TTCTAGCATAAAATACTCCTCCCTA |
| | (SEQ ID NO: 55) |

The following plasmid vectors were constructed:
Plasmid No. 1: pBANak (SEQ ID NO: 47)

It contains the p15A origin of replication, and also the ampR and kanR genes, allowing ampicillin and kanamycin resistance respectively to be conferred. It also contains an expression cassette for Bacillus subtilis phi 3T I methyltransferase, required for methylation of gene-editing vectors prior to transformation in C. acetobutylicum.
Plasmid No. 4: pGRNA_ΔCA_C1340 (SEQ ID NO: 28)

It contains all the elements of pGRNAind (Wasels et al. 2020) with a guide RNA substitution affording CA_C1340 targeting (Oligo-20-mer obtained by hybridization of primers gRNA_CA_C1340_Fwd (SEQ ID NO: 52) and gRNA_CA_C1340_Rev (SEQ ID NO: 53) and incorporated into the plasmid via the “golden gate assembly” technique, after digestion with BsaI. It also contains an editing template allowing inactivation of CA_C1340. This editing template was constructed by cloning the LHA and RHA fragments obtained respectively by amplification with the primer pairs CA_C1340_LHA_Fwd (SEQ ID NO: 48)/CA_C1340_LHA_Rev (SEQ ID NO: 49) and CA_C1340_RHA_Fwd (SEQ ID NO: 50)/CA_C1340_RHA_Rev (SEQ ID NO: 51) from the gDNA of C. acetobutylicum DSM 792. The editing template was cloned between the BamHI and SalI restriction sites.

Transformation of C. acetobutylicum Strains

The plasmids prepared in the NEB 10-beta strain were methylated by transformation into an NEB 10-beta strain also containing the pBANak plasmid. The methylated plasmids thus extracted were then used to transform the C. acetobutylicum strain. Transformation was performed according to the protocol described by Wasels et al. 2020.
Genetic editing of the CA_C1340 gene (SEQ ID NO: 3):

Inactivation of the CA_C1340 gene in ΔCA_C1340 strains was performed by gene editing according to the protocol described by Wasels et al. 2020, respectively using primer pairs CA_C1340_verif_Fwd (SEQ ID NO: 54)/CA_C1340_verif_Rev (SEQ ID NO: 55) during editing validation. The use of other genetic tools, such as those based on homologous recombination or the insertion of mobile genetic elements, may afford mutants with equivalent genotypes, i.e. no longer expressing the CA_C1340 gene product or expressing a non-functional version thereof.

### Results

### Growth Kinetics and Determination of Generation Time

Growth monitoring of wild-type C. acetobutylicum DSM 792 and ΔCA_C1340 strains was performed in quintuplicate on CGM medium containing 30 mM xylose (FIG. 5).

The results reveal an improvement in generation time when growing on xylose as a carbon source. Also, inactivation of the gene encoding the AraR repressor allowed a +152% improvement in generation time relative to the wild-type strain.

### Monitoring of Consumption of a Sugar Mixture

Sugar mixture consumption monitoring was performed in technical triplicates with wild-type C. acetobutylicum DSM 792 and ΔCA_C1340 strains on CGM medium containing 25 mM glucose, 25 mM arabinose and 70 mM xylose (FIG. 6).

The results indicate that inactivation of CA_C1340 afforded faster arabinose consumption kinetics in the presence of glucose than those observed in the wild-type strain.

### Example 3: Strain IFP 968 (LMG P-32703), C. acetobutylicum ΔCA C1340-ΔCA C2613-ΔCA C3673

### Materials and Methods

### Growing Conditions

C. acetobutylicum DSM 792 was grown in 2YTG medium (Tryptone 16 g·L⁻¹, yeast extract 10 g·L⁻¹, glucose 5 g·L⁻¹, NaCl 4 g·L⁻¹) and CGM (KH₂PO₄, 0.75 g; K₂HPO₄, 0.75 g; MgSO₄·7H₂O, 0.40 g; MnSO₄·H₂O, 0.01 g; FeSO₄·7H₂O, 0.01 g; NaCl, 1.0 g; asparagine, 2.0 g; yeast extract, 5.0 g; (NH₄)₂SO₄, 2.0 g; carbon source as specified). Escherichia coli NEB 10-beta was grown in LB medium (Tryptone 10 g·L⁻¹, yeast extract 5 g·L⁻¹, NaCl 10 g·L⁻¹). Solid media were produced by adding 15 g·L⁻¹ of agarose to liquid media. Erythromycin (at concentrations of 40 mg·L⁻¹ in 2YTG medium), chloramphenicol (25 or 12.5 mg L⁻¹ in solid or liquid LB medium respectively), thiamphenicol (15 mg L⁻¹ in 2YTG medium) were used as required.

### Nucleic Acids and Plasmid Vectors

The list of primers used for all the constructs (name/DNA sequence) is detailed below:

| | |
|---|---|
| CA_C1340_LHA Fwd: | AAAAAAGGATCCACCAGAAAAACCAACGCCTAGTA (SEQ ID NO: 48) |
| CA_C1340_LHA_Rev: | AGAAGTAGAAGAATTAAAATTGAAATAATTTATTTAGTCCCTTGCC (SEQ ID NO: 49) |
| CA_C1340 RHA Fwd: | TTTCAATTTTAATTCTTCTACTTCTTCATATTTGTGCT (SEQ ID NO: 50) |
| CA_C1340_RHA Rev: | AAAAAAGTCGACTAATTACTCATTACATACGTTATTTTTCAGT (SEQ ID NO: 51) |
| gRNA_CA_C1340_Fwd: | TCATTTTAAGGTCGATGATTCACA (SEQ ID NO: 52) |
| gRNA_CA_C1340_Rev: | AAACTGTGAATCATCGACCTTAAA (SEQ ID NO: 53) |
| CA_C1340_verif Fwd: | CCTACCAATAGCATCGCCCAAGA (SEQ ID NO: 54) |
| CA_C1340_verif_Rev: | TTCTAGCATAAAATACTCCTCCCTA (SEQ ID NO: 55) |
| CA_C2613 LHA Fwd: | AAAAAAGAATTCATTTGTCCACTTATACCAATTCCT (SEQ ID NO: 31) |
| CA_C2613_LHA_Rev: | TCCTACTAGATACGGCGGAGTAAAATTAGT (SEQ ID NO: 32) |
| CA_C2613 RHA Fwd: | CTCCGCCGTATCTAGTAGGAATCTCCCACT (SEQ ID NO: 33) |
| CA_C2613_RHA Rev: | AAAAAAGTCGACAAAGAGAAAATAAGAGGAATACAAAAG (SEQ ID NO: 34) |
| gRNA_CA_C2613_Fwd: | TCATGTTACACTTGGAACAGGCGT (SEQ ID NO: 35) |
| gRNA_CA_C2613_Rev: | AAACACGCCTGTTCCAAGTGTAAC (SEQ ID NO: 36) |
| CA_C2613_verif Fwd: | TCGGCAGCAAAAACTCCAGT (SEQ ID NO: 42) |
| CA_C2613_verif_Rev: | GCAATTGCTGGCGGAATGAA (SEQ ID NO: 43) |
| CA_C3673_LHA Fwd: | AAAAAAGGATCCAGAAAGACTTGTTACATCTCTAAGT (SEQ ID NO: 37) |
| CA C3673 LHA Rev: | TGATATAGACTTAATTGAACAAGATGTAATTTGAAATTAGT (SEQ ID NO: 38) |
| CA C3673 RHA Fwd: | GTTCAATTAAGTCTATATCAAACATATCGCACCAACC (SEQ ID NO: 39) |
| CA_C3673 RHA Rev: | AAAAAAGTCGACATGATAATGGGAAATATATAGGAACTTT (SEQ ID NO: 40) |
| gRNA_CA_C3673_Fwd: | TCATGGAGTAGCAAGCTCTACAGG (SEQ ID NO: 41) |
| gRNA_CA_C3673_Rev: | AAACCCTGTAGAGCTTGCTACTCC (SEQ ID NO: 42) |
| CA_C3673_verif Fwd: | TACTTCATACATATCTAACGCACTCT (SEQ ID NO: 44) |
| CA_C3673_verif Rev: | AAATAACTTCTACTATGAGACAAGCA (SEQ ID NO: 45) |

The following plasmid vectors were constructed:
Plasmid No. 1: pBANak (SEQ ID NO: 52)

It contains the p15A origin of replication, and also the ampR and kanR genes, allowing ampicillin and kanamycin resistance respectively to be conferred. It also contains an expression cassette for Bacillus subtilis phi 3T I methyltransferase, required for methylation of gene-editing vectors prior to transformation in C. acetobutylicum.
Plasmid No. 4: pGRNA_ΔCA_C1340 (SEQ ID NO: 28)

It contains all the elements of the pGRNAind (Wasels et al. 2020) with a guide RNA substitution allowing CA_C1340 targeting (Oligo-20-mer obtained by hybridization of the primers gRNA_CA_C1340_Fwd (SEQ ID NO: 52) and gRNA_CA_C1340_Rev (SEQ ID NO: 53) and integrated into the plasmid via the “golden gate assembly” technique, after digestion with BsaI. It also contains an editing template allowing inactivation of CA_C1340. This editing template was constructed by cloning the LHA and RHA fragments obtained respectively by amplification with primer pairs CA_C1340_LHA_Fwd (SEQ ID NO: 48)/CA_C1340_LHA_Rev (SEQ ID NO: 49) and CA_C1340_RHA_Fwd (SEQ ID NO: 50)/CA_C1340_RHA_Rev (SEQ ID NO: 51) from the gDNA of C. acetobutylicum DSM 792. The editing template was cloned between the BamHI and SalI restriction sites.
Plasmid No. 2: pGRNA_ΔCA_C2613 (SEQ ID NO: 29)

It contains all the elements of the pGRNAind with a guide RNA substitution allowing CA_C2613 targeting (Oligo-20-mer obtained by hybridization of the primers gRNA_CA_C2613_Fwd (SEQ ID NO: 35) and gRNA_CA_C2613_Rev (SEQ ID NO: 36) and incorporated into the plasmid via the “golden gate assembly” technique, after digestion with BsaI. It also contains an editing template allowing inactivation of CA_C2613. This editing template was constructed by cloning the LHA and RHA fragments obtained respectively by amplification with the primer pairs CA_C2613_LHA_Fwd (SEQ ID NO: 31)/CA_C2613_LHA_Rev (SEQ ID NO: 32) and CA_C2613 RHA_Fwd (SEQ ID NO: 33)/CA_C2613_RHA_Rev (SEQ ID NO: 34) from the gDNA of C. acetobutylicum DSM 792. The editing template was cloned between the BamHI and SalI restriction sites.
Plasmid No. 3: pGRNA_ΔCA_C3673 (SEQ ID NO: 30)

It contains all the elements of the pGRNAind with a guide RNA substitution affording CA_C3673 targeting (Oligo-20-mer obtained by hybridization of the primers gRNA_CA_C3673_Fwd (SEQ ID NO: 41) and gRNA_CA_C3673_Rev (SEQ ID NO: 42) and incorporated into the plasmid by the golden gate assembly technique, after digestion with BsaI. It also contains an editing template allowing inactivation of CA_C3673. This editing template was constructed by cloning the LHA and RHA fragments obtained respectively by amplification with the primer pairs CA_C3673_LHA_Fwd (SEQ ID NO: 37)/CA_C3673_LHA_Rev (SEQ ID NO: 38) and CA_C3673_RHA_Fwd (SEQ ID NO: 39)/CA_C3673_RHA_Rev (SEQ ID NO: 40) from the gDNA of C. acetobutylicum DSM 792. The editing template was cloned between the BamHI and SalI restriction sites.

Transformation of C. acetobutylicum Strains

The plasmids prepared in the NEB 10-beta strain were methylated by transformation into an NEB 10-beta strain also containing the pBANak plasmid. The methylated plasmids thus extracted were then used to transform the C. acetobutylicum strain. Transformation was performed according to the protocol described by Wasels et al. 2020.
Genetic editing of CA_C2613 (SEQ ID NO: 1), CA_C3673 (SEQ ID NO: 2) and/or CA_C1340 (SEQ ID NO: 3) genes:

Inactivations of the CA_C1340, CA_C2613 and CA_C3673 genes in the ΔCA_C1340-ΔCA_C2613-ΔCA_C3673 strain were performed by gene editing according to the protocol described by Wasels et al. 2020, respectively using primer pairs CA_C1340_verif_Fwd (SEQ ID NO: 54)/CA_C1340_verif_Rev (SEQ ID NO: 55); CA_C2613_verif_Fwd (SEQ ID NO: 43)/CA_C2613_verif_Rev (SEQ ID NO: 44); CA_C3673_verif_Fwd (SEQ ID NO: 45)/CA_C3673_verif_Rev (SEQ ID NO: 46) during editing validation. The use of other genetic tools, such as those based on homologous recombination or the insertion of mobile genetic elements, may afford mutants possessing equivalent genotypes, i.e. no longer expressing the products of the CA_C1340, CA_C2613 and/or CA_C3673 genes, or expressing a non-functional version thereof.

### Results

### Growth Kinetics and Determination of Generation Time

Growth monitoring of wild-type C. acetobutylicum DSM 792, and ΔCA_C1340 ΔCA_C2613 ΔCA_C3673 strains was performed in quintuplicate on CGM medium containing 30 mM xylose (FIG. 5).

The results reveal an improvement in generation time when growing on xylose as a carbon source. Also, joint inactivation of the genes encoding the AraR and XylR repressors allowed a +171% improvement in generation time relative to the wild-type strain.

### Monitoring of Consumption of a Sugar Mixture

Sugar mixture consumption monitoring was performed in technical triplicates with wild-type C. acetobutylicum DSM 792 and ΔCA_C1340 ΔCA_C2613 ΔCA_C3673 strains, on CGM medium containing 25 mM glucose, 25 mM arabinose and 70 mM xylose (FIG. 6).

The results indicate that the simultaneous co-assimilation of arabinose and glucose is made possible by the joint inactivation of CA_C1340 and the CA_C2613 and CA_C3673 genes.

The catabolic repression preventing xylose co-assimilation in the presence of the other two sugars is also maintained. This unexpected result indicates that synergism between joint inactivation of the genes encoding the AraR and XylR repressors allows catabolic repression of arabinose to be lifted, while at the same time maintaining it for xylose.

### CONCLUSIONS

In the course of this work, the inventors succeeded in demonstrating the value of joint inactivation of the genes encoding the AraR and XylR repressors in the C. acetobutylicum DSM 792 strain, thus describing how the combination of these genetic modifications allows the production of a C. acetobutylicum strain performing glucose/arabinose co-assimilation and having accelerated growth on xylose alone. Inactivation of the araR gene alone does not allow co-assimilation of arabinose and glucose, which is only obtained when copies of the genes encoding the XylR repressor are also inactivated. Furthermore, inactivation of both copies of the gene encoding XylR is not sufficient to significantly reduce the generation time of the microorganisms when growing on a medium containing xylose as the sole carbon source. Inactivation of the gene encoding AraR is therefore necessary.

This is an important result, as this strain naturally suffers from an inability to assimilate pentoses and hexoses such as glucose, arabinose and xylose simultaneously, drastically impacting performance when fermenting on substrates composed of a mixture of these sugars, such as the industrial substrates of biomass-based sugar production processes.

### REFERENCES

Aristilde, Ludmilla; Lewis, Ian A.; Park, Junyoung O.; Rabinowitz, Joshua D. (2014) Hierarchy in Pentose Sugar Metabolism in Clostridium acetobutylicum. In: Applied and Environmental Microbiology, vol. 81, No. 4, pp. 1452-1462. DOI: 10.1128/aem.03199-14.
Ezeji, Thaddeus; Blaschek, Hans P. (2008) Fermentation of dried distillers' grains and solubles (DDGS) hydrolysates to solvents and value-added products by solventogenic clostridia. In: Bioresource Technology, vol. 99, No. 12, pp. 5232-5242. DOI: 10.1016/j.biortech.2007.09.032.
Ezeji, Thaddeus; Qureshi, Nasib; Blaschek, Hans P. (2007) Butanol production from agricultural residues. Impact of degradation products on Clostridium beijerinckii growth and butanol fermentation. In: Biotechnology and Bioengineering, vol. 97, No. 6, pp. 1460-1469. DOI: 10.1002/bit.21373.
Grimmler, Christina; Held, Claudia; Liebl, Wolfgang; Ehrenreich, Armin (2010) Transcriptional analysis of catabolite repression in Clostridium acetobutylicum growing on mixtures of D-glucose and D-xylose. In: Journal of Biotechnology, vol. 150, No. 3, pp. 315-323. DOI: 10.1016/j.jbiotec.2010.09.938.
Gu, Yang; Ding, Yi; Ren, Cong; Sun, Zhe; Rodionov, Dmitry A.; Zhang, Weiwen et al. (2010) Reconstruction of xylose utilization pathway and regulons in Firmicutes. In: BMC Genomics, vol. 11, pp. 255. DOI: 10.1186/1471-2164-11-255.
Hu, Shiyuan; Zheng, Huajun; Gu, Yang; Zhao, Jingbo; Zhang, Weiwen; Yang, Yunliu et al. (2011) Comparative genomic and transcriptomic analysis revealed genetic characteristics related to solvent formation and xylose utilization in Clostridium acetobutylicum EA 2018. In: BMC Genomics, vol. 12, No. 1. DOI: 10.1186/1471-2164-12-93.
Keis, S.; Shaheen, R.; Jones, D. T. (2001) Emended descriptions of Clostridium acetobutylicum and Clostridium beijerinckii, and descriptions of Clostridium saccharoperbutylacetonicum sp. nov. and Clostridium saccharobutylicum sp. nov. In: International Journal of Systematic and Evolutionary Microbiology, vol. 51, No. 6, pp. 2095-2103. DOI: 10.1099/00207713-51-6-2095.
Qureshi, N.; Li, X.-L.; Hughes, S.; Saha, B. C.; Cotta, M. A. (2006) Butanol production from corn fiber xylan using Clostridium acetobutylicum. In: Biotechnology Progress, vol. 22, No. 3, pp. 673-680. DOI: 10.1021/bp050360w.
Qureshi, Nasib; Ezeji, Thaddeus C.; Ebener, Jennifer; Dien, Bruce S.; Cotta, Michael A.; Blaschek, Hans P. (2008) Butanol production by Clostridium beijerinckii. Part I. Use of acid and enzyme hydrolyzed corn fiber. In: Bioresource Technology, vol. 99, No. 13, pp. 5915-5922. DOI: 10.1016/j.biortech.2007.09.087.
Servinsky, M. D. Arabinose-induced catabolite repression as a mechanism for pentose hierarchy control in Clostridium acetobutylicum ATCC 824.
Servinsky, M. D.; Germane, K. L.; Liu, S.; Kiel, J. T.; Clark, A. M.; Shankar, J.; Sund, C. J. (2012) Arabinose is metabolized via a phosphoketolase pathway in Clostridium acetobutylicum ATCC 824. In: Journal of Industrial Microbiology & Biotechnology, vol. 39, No. 12, pp. 1859-1867. DOI: 10.1007/s10295-012-1186-x.
Servinsky, Matthew D.; Kiel, James T.; Dupuy, Nicole F.; Sund, Christian J. (2010) Transcriptional analysis of differential carbohydrate utilization by Clostridium acetobutylicum. In: Microbiology (Reading, England), vol. 156, No. Pt 11, pp. 3478-3491. DOI: 10.1099/mic.0.037085-0.
Wasels, François; Chartier, Gwladys; Hocq, Rémi; Lopes Ferreira, Nicolas; Julia Pettinari, M. (2020) A CRISPR/Anti-CRISPR genome editing approach underlines the synergy of butanol dehydrogenases in Clostridium acetobutylicum DSM 792. In: Applied and Environmental Microbiology, vol. 86, No. 13, p. 4823. DOI: 10.1128/AEM.00408-20.
Zhiqiang Wen, Rodrigo Ledesma-Amaro, MinruiLLu, Mingjie Jin, and Sgheng Yang (2020) Metabolic engineering of Clostridium cellulovorans to improve butanol production by consolidated bioprocessing. ACS Synth. Biol. 9, 304-315.
Xiao, Han; Li, Zhilin; Jiang, Yu; Yang, Yunliu; Jiang, Weihong; Gu, Yang; Yang, Sheng (2012)
Metabolic engineering of D-xylose pathway in Clostridium beijerinckii to optimize solvent production from xylose mother liquid. In: Metabolic Engineering, vol. 14, No. 5, pp. 569-578. DOI: 10.1016/j.ymben.2012.05.003.
Zhang, Lei; Leyn, Semen A.; Gu, Yang; Jiang, Weihong; Rodionov, Dmitry A.; Yang, Chen (2012) Ribulokinase and transcriptional regulation of arabinose metabolism in Clostridium acetobutylicum. In: Journal of Bacteriology, vol. 194, No. 5, pp. 1055-1064. DOI: 10.1128/JB.06241-11.

1-17. (canceled)

18. A genetically modified bacterium belonging to the genus Clostridium, characterized in that it does not express the products of the xylR and araR genes, or in that it expresses non-functional versions thereof.

19. The genetically modified bacterium according to claim 18, characterized in that the bacterium is a solventogenic bacterium or is C. acetobutylicum.

20. The genetically modified bacterium according to claim 19, characterized in that i) the bacterium belongs to the species C. acetobutylicum and does not express the gene products of sequences SEQ ID NO: 1 (CA_C2613) or a sequence at least 95% homologous thereto, of SEQ ID NO: 2 (CA_C3673) or a sequence at least 95% homologous thereto, and of SEQ ID NO: 3 (CA_C1340) or a sequence at least 95% homologous thereto, or ii) it expresses non-functional versions of said expression products.

21. The genetically modified bacterium according to claim 18, characterized in that it is capable of simultaneously fermenting at least one hexose and one pentose.

22. The genetically modified bacterium according to claim 18, characterized in that it is capable of simultaneously fermenting the carbon-based components of a substrate comprising at least two carbon components chosen from glucose, arabinose, xylose, mannose and galactose, preferably chosen from glucose, arabinose and xylose.

23. The genetically modified bacterium according to claim 18, characterized in that it grows faster than a bacterium expressing a functional AraR protein, or a mixture of functional XylR and AraR proteins, on a medium comprising xylose as the sole carbon source.

24. The genetically modified bacterium according to claim 18, characterized in that the bacterium is the strain IFP 968 registered on 13 Jun. 2022 under deposit number LMG P-32703 at the BCCM-LMG Collection.

25. A fermentation process comprising contacting a carbon source with a bacterium as described in claim 18.

26. The fermentation process according to claim 25, wherein the process simultaneously ferments at least one hexose and one pentose or the carbon-based elements of a substrate comprising at least two carbon-based elements selected from glucose, arabinose, xylose, mannose and galactose.

27. The fermentation process according to claim 25, wherein the process simultaneously ferments at least one hexose and one pentose or the carbon-based elements of a substrate comprising at least two sugars selected from glucose, arabinose and xylose.

28. The fermentation process according to claim 25, wherein the process produces a solvent, a biofuel, or a mixture of solvents by simultaneously fermenting at least one hexose and one pentose or the carbon-based elements of a substrate comprising at least two carbon-based elements selected from glucose, arabinose, xylose, mannose and galactose.

29. A process for producing a sugar or a biobased molecule comprising contacting a bacterium as described in claim 18 with i) a lignocellulosic biomass, ii) the product of a particular dedicated crop such as an energy crop, a perennial forage crop, a herbaceous crop, a shrub crop, or iii) 1G substrate(s) derived from one or more sugar-yielding, cereal or oil-yielding plants, or iv) a mixture thereof.

30. A process for producing a recombinant bacterium according to claim 18, comprising the deletion or inactivation of the xyIR and araR genes so as to prevent or reduce the expression of functional XylR and AraR proteins.

31. The process according to claim 30, characterized in that the genetically modified bacterium not expressing the xylR or araR gene product, or expressing a non-functional version of the xylR or araR gene product, is the strain IFP 967 registered on 13 Jun. 2022 under deposit number LMG P-32702 at the BCCM-LMG collection.

32. A genetically modified bacterium belonging to the genus Clostridium obtained by the process according to claim 30, characterized in that said bacterium is a bacterium which does not express the products of the xylR and araR genes, or in that it expresses non-functional versions thereof.

33. A kit for producing a solvent, a sugar or a biobased molecule comprising a genetically modified bacterium belonging to the genus Clostridium according to claim 18 and a storage medium for said bacterium.
